(19) United States
(12) Patent Application Publication
(10) Pub. No.: US 2026/0351383 A1
(43) Pub. Date: Nov. 5, 2026

(54) DEVICE FOR PREPARING OF A MIXTURE OF SUPPLEMENTS

(71) Applicant: Agen Com Pte. Ltd., SINGAPORE (SG)

(72) Inventors: Vladimir SITNIKOV, Singapore (SG); Serg BELL, Singapore (SG)

(21) Appl. No.: 19/461,855

(22) Filed: Jan. 28, 2026

Related U.S. Application Data
(63) Continuation-in-part of application No. 19/003,472, filed on Dec. 27, 2024.

(51) Int. Cl. G16H 20/60; B65D 83/04; G16H 40/63

(52) U.S. Cl. CPC G16H 20/60; B65D 83/04; G16H 40/63

(57) ABSTRACT

A device includes a plurality of ingredient interfaces each couplable to a replaceable container. The device includes a hydraulic system for pumping liquids with metering pumps. The device includes an electronic controller configured to: receive an instruction to prepare a mixture based on a general recipe, detect one or more replaceable containers currently coupled to at least one of the plurality of ingredient interfaces, determine an ingredient identifier list associated with the one or more replaceable containers and associated properties of ingredients in the ingredient identifier list; and in response to the instruction, actuate selected metering pumps of the hydraulic system to deliver ingredient volumes from the one or more replaceable containers in accordance with the general recipe and initiate a rinsing flow through the hydraulic system toward an outlet.

### CROSS-REFERENCE TO RELATED APPLICATIONS

This application is a continuation-in-part of United States Non-Provisional Application No. 19/003,472, filed Dec. 27, 2024, which is herein incorporated by reference.

### FIELD OF TECHNOLOGY

The present disclosure relates to a personalized dietary supplements, and devices and systems configured to produce the same using machine learning and/or other parameters, techniques and algorithms set forth here.

### BACKGROUND

In the U.S. and elsewhere, consumers are increasingly turning to dietary supplements (e.g., containing vitamins, minerals, probiotics, and other functional components) to improve their health and well-being, and to help achieve personal goals such as weight loss. Consumers typically obtain individual, pre-packaged dietary supplements and simply ingest a recommended amount daily, or according to a generic dosage schedule set by the manufacturer or recommended by a medical professional. Some personalized solutions have been proposed and/or commercialized. However, these systems typically lack extensive customization options and are not designed to account for the combination or mixture of multiple ingredients. Moreover, these systems typically consist of nothing more than a regimen for the user to follow by manually preparing recommended amounts of multiple ingredients. As such, prior systems do not often truly personalized dietary supplements, nor do they provide a simple and efficient system for preparing personalized dietary supplements.

### SUMMARY

In one exemplary aspect, the techniques described herein relate to a device for preparing of a mixture of supplements, including: a plurality of ingredient interfaces each couplable to a replaceable container; a hydraulic system for pumping liquids with metering pumps; and an electronic controller configured to: receive an instruction to prepare a mixture based on a general recipe; detect one or more replaceable containers currently coupled to at least one of the plurality of ingredient interfaces; determine an ingredient identifier list associated with the one or more replaceable containers and associated properties of ingredients in the ingredient identifier list; and in response to the instruction, actuate selected metering pumps of the hydraulic system to deliver ingredient volumes from the one or more replaceable containers in accordance with the general recipe and initiate a rinsing flow through the hydraulic system toward an outlet.

In some aspects, the techniques described herein relate to a device, wherein the associated properties include one or more of: physical properties including at least viscosity, chemical properties, and a current remaining volumes of the ingredients in the one or more replaceable containers.

In some aspects, the techniques described herein relate to a device, wherein the hydraulic system includes: tubing forming a common conduit and branch lines from the metering pumps, the common conduit extending from a water valve to an outlet nozzle disposed at a front portion of the device above a cup platform; one-way check valves downstream of each respective metering pump to prevent backflow toward the respective metering pump, and additional one-way valves disposed in the common conduit upstream of each junction where a branch line connects to prevent reverse flow into other lines; and a side-mounted reservoir with a lid, installable and removable by sliding sideways or by pulling up relative to the device.

In some aspects, the techniques described herein relate to a device, wherein the hydraulic system executes: a cleaning cycle using a special cleaning capsule to flush and sanitize the hydraulic system including the common conduit and the outlet nozzle.

In some aspects, the techniques described herein relate to a device, wherein the selected metering pumps are actuated based on a dosing scheme that indicates a time of actuation of at least one metering pump based on physical properties of a corresponding ingredient.

In some aspects, the techniques described herein relate to a device, wherein each ingredient interface of the plurality of ingredient interfaces includes: a pair of piercing needles fixed to a plastic grid that serves as a container holder, the needles projecting into a respective cell of the plastic grid; wherein the needles are fluidically connected such that a first needle is coupled to a first metering pump to extract liquid from a container and a second needle is coupled to an air path to admit air into the container; wherein the needles are positioned to engage two internal valves of a cooperating container, one valve providing a liquid outlet and another value providing an air inlet; cell-level orientation features including lateral grooves in a lower third of each cell that key container insertion in only one orientation relative to the needles; and a push-to-open cover for each cell, biased by an internal pusher to keep the cell closed in an absence of a container to protect the needles from exposure and accidental contact, the push-to-open cover opens upon being pressed for container insertion.

In some aspects, the techniques described herein relate to a device, wherein the device is compatible with a factory-sealed, single-use container having a parallelepiped body with a lid, an affixed NFC tag, and two internal one-way valves on an inner surface respectively engaged by the needles.

In some aspects, the techniques described herein relate to a device, further including: a structural frame; a rear, upper internal bracket supporting a power supply; a rear power inlet; a rear on/off switch; and a power cord connecting the rear power inlet to the power supply.

In some aspects, the techniques described herein relate to a device, further including: a printed circuit board mounted within a central space of the structural frame and connected to the power supply at an upper portion; a body of the device includes of a container side and a part for installing a water reservoir and covering a central part when the water reservoir is removed; a front display mounted to the structural frame and surrounded by a snap-fit bezel; and a cup platform disposed below an outlet nozzle, the cup platform seated in grooves and retained by a weight of the cup platform.

In some aspects, the techniques described herein relate to a device, wherein the electronic controller includes a plurality of controllers including: a real-time operating system (RTOS) controller on a printed circuit board configured to operation of the metering pumps and a rinse function; a front display controller in communication with the RTOS controller and configured to issue preparation commands; and wherein the metering pumps are electrically connected to the printed circuit board by connectors.

In some aspects, the techniques described herein relate to a device, further including: a wireless network interface configured to connect to a Wi-Fi network and a display function configured to present a QR code for pairing the device with a user profile via a mobile application; and an NFC reader located at a marked area adjacent a container holder and configured to read NFC tags on containers, the device being configured to transmit information about installed containers to a server, to receive and present a server-provided slot assignment for each container, and to receive preparation formulas specifying slot identifiers, pump activation durations, and pump drive voltages.

In some aspects, the techniques described herein relate to a method for preparing of a mixture of supplements using a dispensing device, including: receiving, by an electronic controller of the dispensing device, an instruction to prepare a mixture based on a general recipe; detecting, by the electronic controller, one or more replaceable containers currently coupled to at least one of a plurality of ingredient interfaces; determining, by the electronic controller, an ingredient identifier list associated with the one or more replaceable containers and associated properties of ingredients in the ingredient identifier list; and in response to the instruction, actuating selected metering pumps of a hydraulic system of the dispensing device to deliver ingredient volumes from the one or more replaceable containers in accordance with the general recipe and initiate a rinsing flow through the hydraulic system toward an outlet of the dispensing device.

In some aspects, the techniques described herein relate to a method, wherein the associated properties include one or more of: physical properties including at least viscosity, chemical properties, and a current remaining volumes of the ingredients in the one or more replaceable containers.

In some aspects, the techniques described herein relate to a method, wherein the hydraulic system includes: tubing forming a common conduit and branch lines from the metering pumps, the common conduit extending from a water valve to an outlet nozzle disposed at a front portion of the method above a cup platform; one-way check valves downstream of each respective metering pump to prevent backflow toward the respective metering pump, and additional one-way valves disposed in the common conduit upstream of each junction where a branch line connects to prevent reverse flow into other lines; and a side-mounted reservoir with a lid, installable and removable by sliding sideways or by pulling up relative to the dispensing device.

In some aspects, the techniques described herein relate to a method, wherein the hydraulic system executes: a cleaning cycle using a special cleaning capsule to flush and sanitize the hydraulic system including the common conduit and the outlet nozzle.

In some aspects, the techniques described herein relate to a method, wherein the selected metering pumps are actuated based on a dosing scheme that indicates a time of actuation of at least one metering pump based on physical properties of a corresponding ingredient.

In some aspects, the techniques described herein relate to a method, wherein each ingredient interface of the plurality of ingredient interfaces includes: a pair of piercing needles fixed to a plastic grid that serves as a container holder, the needles projecting into a respective cell of the plastic grid; wherein the needles are fluidically connected such that a first needle is coupled to a first metering pump to extract liquid from a container and a second needle is coupled to an air path to admit air into the container; wherein the needles re positioned to engage two internal valves of a cooperating container, one valve providing a liquid outlet and another value providing an air inlet; cell-level orientation features including lateral grooves in a lower third of each cell that key container insertion in only one orientation relative to the needles; and a push-to-open cover for each cell, biased by an internal pusher to keep the cell closed in an absence of a container to protect the needles from exposure and accidental contact, the push-to-open cover opens upon being pressed for container insertion.

In some aspects, the techniques described herein relate to a method, wherein the dispensing device is compatible with a factory-sealed, single-use container having a parallelepiped body with a lid, an affixed NFC tag, and two internal one-way valves on an inner surface respectively engaged by the needles.

In some aspects, the techniques described herein relate to a method, further including: a structural frame; a rear, upper internal bracket supporting a power supply; a rear power inlet; a rear on/off switch; and a power cord connecting the rear power inlet to the power supply.

In some aspects, the techniques described herein relate to a method, further including: a printed circuit board mounted within a central space of the structural frame and connected to the power supply at an upper portion; a body of the dispensing device includes of a container side and a part for installing a water reservoir and covering a central part when the water reservoir is removed; a front display mounted to the structural frame and surrounded by a snap-fit bezel; and a cup platform disposed below an outlet nozzle, the cup platform seated in grooves and retained by a weight of the cup platform.

The above simplified summary of example aspects serves to provide a basic understanding of the present disclosure. This summary is not an extensive overview of all contemplated aspects, and is intended to neither identify key or critical elements of all aspects nor delineate the scope of any or all aspects of the present disclosure. Its sole purpose is to present one or more aspects in a simplified form as a prelude to the more detailed description of the disclosure that follows. To the accomplishment of the foregoing, he one or more aspects of the present disclosure include the features described and exemplarily pointed out in the claims.

### BRIEF DESCRIPTION OF THE DRAWINGS

The accompanying drawings, which are incorporated into and constitute a part of this specification, illustrate one or more example aspects of the present disclosure and, together with the detailed description, serve to explain their principles and implementations.

FIG. 1 is block diagram showing aspects of an exemplary system for generating a personalized dietary supplement according to the present disclosure.

FIG. 2 is a block diagram showing aspects of an exemplary system for generating a personalized dietary supplement according to the present disclosure. This figure highlights potential data sources and the layout of modules used in some aspects of the systems described herein.

FIG. 3 is a block diagram showing aspects of an exemplary system for generating a personalized dietary supplement according to the present disclosure. This figure highlights potential data sources and the layout of modules used in some aspects of the systems described herein.

FIG. 4 is a flow chart showing an exemplary method for generating a personalized dietary supplement according to the present disclosure.

FIG. 5 is a diagram of a personalized dietary supplement dispenser.

FIG. 6 is a diagram of personalized dietary supplement dispenser with a protruding capsule.

FIG. 7a is a diagram of a set of internal components of personalized dietary supplement dispenser.

FIG. 7b is a diagram of a frame that supports a plurality of capsules in personalized dietary supplement dispenser.

FIG. 8 is a diagram of the piping and hydraulic system of personalized dietary upplement dispenser.

FIG. 9 is a diagram of piping in a branch of the hydraulic system.

FIG. 10 is a diagram depicting a plurality of ingredient interfaces.

FIG. 11 is a diagram of an exemplary capsule.

FIG. 12 is a diagram that shows liquid flow within personalized dietary supplement dispenser.

FIG. 13 is a diagram that shows a first user interface output on a display of personalized dietary supplement dispenser.

FIG. 14 is a diagram that shows a second user interface output on a display of personalized dietary supplement dispenser.

FIG. 15 is a diagram that shows a third user interface output on a display of personalized dietary supplement dispenser.

FIG. 16 is a diagram that shows a fourth user interface output on a display of personalized dietary supplement dispenser.

FIG. 17 is a diagram that shows a fifth user interface output on a display of personalized dietary supplement dispenser.

FIG. 18 is a diagram that shows a sixth user interface output on a display of personalized dietary supplement dispenser.

FIG. 19 is a flow diagram of a method for preparing of a mixture of supplements using a dispensing device.

FIG. 20 is a flow diagram of a method for preparing and delivering a supplement mixture using a dispensing device.

FIG. 21 presents an example of a general-purpose computer system on which aspects of the present disclosure can be implemented.

### DETAILED DESCRIPTION

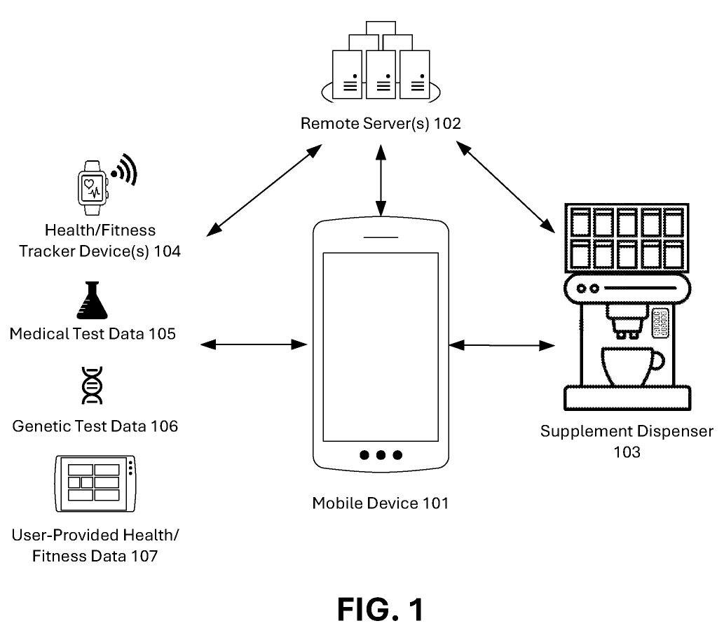

FIG. 1 is block diagram showing aspects of an exemplary system for generating a personalized dietary supplement according to the present disclosure. In this case, the system 100 includes a mobile device 101, one or more remote servers 102, a supplement dispenser 103, and several sources of data and/or parameters that may be used to generate personalized supplements, 104-107.

As illustrated by this figure, a user may be able to interact with the supplement dispenser 103 and/or the other components of the system 100 using a mobile device 101. In this example, the mobile device 101 is a user’s smart phone. In other aspects, the mobile device 101 may comprise a wearable device (e.g., a smart watch, smart glasses, etc.), a tablet, a laptop, or a dedicated controller for the device. It is understood that in some aspects the user may be able to interact with the components of the system 100 using a device that it not typically mobile (e.g., a desktop computer). Thus, any references to systems that utilize a mobile device 101 should be understood as also contemplating alternatives using fixed or non-mobile devices as well, in alternative aspects. Regardless, the mobile device 101 (or other means of control) may be configured to execute software code designed to generate a graphical user interface (GUI) that allows the user to interact with the supplement dispenser 103. For example, a GUI may allow a user to view the status of the supplement dispenser 103, and/or the current quantity, amount, concentration, and/or volume of one or more dietary supplement components stored in the supplement dispenser 103. In some aspects, the GUI may allow a user to view, select, and/or customize dietary supplement recipes stored on the supplement dispenser 103 or on a remote server 102 (e.g., a database of recipes provided by the manufacturer of the supplement dispenser 103 or third-party databases). For example, the GUI may allow a user to select a dietary supplement recipe stored in memory of the supplement dispenser 103 and to modify the recipe my selecting one or more dietary supplement components to add or remove, or by adjusting the amount, quantity, concentration, or volume of one or more dietary supplement components.

In some aspects, the dietary supplement components may be stored in capsules or containers having one or more human and/or machine-readable labels, tags, or chips. For example, a container or capsule may comprise a near-field communication (NFC) or other wirelessly readable tag that stores information about the manufacturer of a dietary supplement component, or production-related information (e.g., series, date of manufacture, expiration date, etc.) or a unique identifier. The personal dietary supplement dispenser 103 may be configured to wirelessly read this NFC (or other wirelessly-readable tag) in order to validate the authenticity of dietary supplement components, and optionally to block the use of counterfeit, unidentified, and/or non-validated dietary supplement components. In some aspects, the human and/or machine-readable labels, tags, or chips may describe information about the amount, level, or concentration of the dietary supplement component in a container or capsule. Other information may also be described, e.g., the time since first (or a most recent) use of the component may be recorded, and the storage temperature of the component may be tracked.

In some aspects, the human and/or machine-readable labels, tags, or chips may be editable (e.g., by the dietary supplement dispenser, or by an app or device (e.g., by an app run on a mobile device of the user, or via a computer or electronic device operated by the user). For example, the personal dietary supplement dispenser may be allowed to read a parameter indicative of an initial amount, level, or concentration of a component, and to record to the label, tag, or chip a new amount, level, or concentration after using a portion of that component in a formula. The reading and writing process may be performed using, e.g., a wireless means of communication such as NFC or Bluetooth. In some aspects, a personal dietary supplement dispenser 103 may comprise 1, 2, 3, 4, or 5 NFC readers (or reader/writer elements) positioned at one or more locations in or on the device. It is further envisioned that the personal dietary supplement dispenser may include one or more NFC readers that allow a user to authorize the generation of a personalized dietary supplement via a one-touch process. For example, a user may be able to authorize the device to proceed with preparing a personalized dietary supplement by tapping an NFC reader interface on the device with a smartphone, key fob, card, or other NFC-compatible device.

In some aspects, the supplement dispenser 103 may receive dietary supplement recipes from a remote server 102 that has been configured to generate dietary supplement recipes for a user. As shown by FIG. 1, the remote server 102 may communicate with various data sources, including without limitation health/fitness tracker devices 104, medical test data 105 sources, genetic test data 106 sources, and user-provided health/fitness data 107. These data sources may comprise, e.g., one or more third-party database, one or more internal databases maintained by the operator of the remote server 102 or the manufacturer of the supplement dispenser 103, or real-time or historic data stored or generated by one or more devices or sensors (e.g., wearable fitness trackers). The remote server 102 may be configured to generate personalized dietary supplement recipes using any combination of these data sources (or any other data sources described herein), using one or more algorithms, predetermined or user-selected parameters, and/or machine learning techniques.

In some aspects, the remote server 102 may store a library of existing recipes, e.g., collected from books or the internet (e.g., webpages run by “influencers” and other online personalities), or designed by medical or health professionals, and may generate a personalized dietary supplement recipe for a given user by customizing one of these existing templates. For example, a user may use the GUI of the mobile device 101 to indicate that they desire to achieve a particular goal (e.g., weight loss), or to increase their consumption of a given dietary supplement component, or to indicate that a given dietary supplement should never be included in a recipe (e.g., due to personal preferences or allergies). The remote server 102 may take any such parameters into account and modify an existing recipe template accordingly.

Similarly, the remote server 102 may take into account health/fitness tracker device 104 data (e.g., real-time or historical data regarding one or more physiological or health-related parameters, or activity-related data, for the user). For example, data from a health/fitness tracker device 104 may comprise data from a smart watch or other wearable device, and may show that a user is engaged in a rigorous physical activity or exercise. In that case, the remote server 102 may account for this data by, e.g., increasing the calories or providing additional electrolytes, in a dietary supplement recipe for the user. Medical test data 105 and genetic test data 106 may also be utilized. For example, medical test data 105 may comprise the results of a recent diagnostic panel showing that the user is deficient in a given nutrient or vitamin (e.g., the user may have low iron levels). The remote server 102 may ccount for this data by, e.g., by adding iron or increasing the concentration of iron in a dietary supplement recipe for the user. Genetic test data 106 may provide similar insights, e.g., such data may reveal that a user has a genetic signature associated with a given health and/or fitness state or outcome, which the remote sever 102 can account for when designing and/or modifying dietary supplement recipes for that user. For example, a user may have a gene that results in slow metabolism, reduced immune system functionality, or another negative characteristic, which can be corrected or mitigated by consumption of a particular dietary supplement component.

In some aspects, the remote server 102 may be configured to account for user-provided health and/or fitness data 107. Such data may be obtained from the user, e.g., via a questionnaire provided in the GUI of an application executed on the mobile device 101, or otherwise made available to the user. For example, the GUI may allow a user to input historical health and/or fitness data (e.g., information about the user’s health, weight, activity level, goals). The remote server 102 may account for this data when formulating or modifying dietary supplement recipes.

In some aspects, the remote server 102 may generate a personalized dietary supplement recipe for a user using one or more algorithms, statistical techniques, or using artificial intelligence (AI) or machine learning (ML) models. For example, an AI/ML model for assessing a health and/or fitness state of the user may be trained using biometric, dietary, health and/or fitness data obtained from or associated with the user, and optionally using historical data from other users. Such models may be trained, e.g., to recognize deficiencies in a user’s diet that can be corrected using a personalized dietary supplement, or dietary supplement components that should be added to a user’s personalized dietary supplement in order to help the user achieve a desired goal (e.g., weight loss, muscle development, improved cardiovascular health, or any other goal described herein). ML models may also be used to design or modify a dietary supplement recipe for the user. For example, the remote server 102 may be configured to generate a set of candidate dietary supplement recipes for a user, and the set can be scored using statistical techniques, or AI/ML algorithms or models in order to identify the best recipe for a user. The best recipe may omprise, e.g., a recipe that is determined based on a scoring metric to be most likely to (a) result in a desired health or fitness outcome or goal selected by the user (e.g., weight loss), or (b) improve the state of the user’s health and/or fitness with respect to one or more physiological parameters or activities (e.g., an improvement to the quality of the user’s sleep or metabolism). For example, candidate recipes may be scored using a model that was previously trained using historical biometric, dietary, health and/or fitness data for a population of users (e.g., where the trained model recognizes. In this case, the pre-trained model may be used to recognize correlations between the historical biometric, health and/or fitness data, and dietary supplement consumption data, for the users in that population. The trained model may reveal, e.g., that users that consume a higher quantity, amount, concentration, or volume or a given dietary supplement component consistently experienced a particular change in one or more physiological parameters, or a particular health and/or fitness outcome (e.g., a pattern may become evident showing that regular consumption of a given vitamin results in a favorable reduction in blood pressure, or a tendency for a user to experience weight loss, muscle gain, or another positive health and/or fitness outcome described herein).

Note that while the foregoing examples contemplate that the remote server 102 may be configured to perform AI/ML-related analysis, in alternative aspects this functionality may be performed by the supplement dispenser 103. The decision to have this functionality performed locally on device or remotely may vary among different implementations.

Various statistical techniques, algorithms, and AI/ML models may be implemented by the systems and devices described herein, when generating or scoring dietary supplement recipes.

In some aspects, the AI/ML models implemented by the remote server 102 (or, in alternative aspects, by the supplement dispenser 103) may comprise one or more supervised learning algorithms configured to map user-specific data to personalized dietary supplement recipes. For example, such models may include, without limitation, linear or logistic regression models, decision tree or random forest models, gradient-boosted ecision tree models (e.g., XGBoost-type models), support vector machine models, artificial neural network models (including deep learning architectures), Bayesian models, or ensemble models that combine the outputs of multiple underlying algorithms. In some aspects, unsupervised learning techniques (e.g., clustering algorithms such as k-means, hierarchical clustering, or Gaussian mixture models) may be used to segment users into groups having similar biometric, dietary, health and/or fitness profiles, and the remote server 102 may select or bias dietary supplement recipes for a current user based on recipes historically determined to be effective for users in the same cluster. In other aspects, reinforcement learning techniques may be utilized, wherein the system 100 iteratively adjusts dietary supplement recipes over time in response to feedback (e.g., subsequent changes in biometric data, self-reported user satisfaction, or achievement of goals), and learns a policy that selects dietary supplement components and dosages that maximize a reward signal associated with improvements in one or more health and/or fitness outcomes.

In one exemplary aspect, the remote server 102 may implement a supervised gradient-boosted decision tree model that is trained to predict one or more outcome scores associated with candidate dietary supplement recipes. The training dataset for this model may include historical biometric, dietary, health and/or fitness data for a population of users (e.g., heart rate, blood pressure, resting heart rate, sleep quality scores, activity levels, step counts, VO₂ max, body weight, body composition, blood panel results, or genetic markers), as well as data describing prior dietary supplement recipes consumed by those users (e.g., vectors indicating the presence or absence, and/or the quantity, amount, concentration, or volume, of each dietary supplement component in a recipe), which may be derived from data stored by the supplement dispenser 103, the mobile device 101, or third-party databases. Additional inputs to the model may include user-provided health/fitness data 107 (e.g., age, sex, dietary restrictions, allergies, user-selected goals such as weight loss or muscle gain) and real-time or historical data from health/fitness tracker devices 104, medical test data 105, and genetic test data 106. The training labels or targets for the model may comprise one or more quantitative or categorical measures of health and/or fitness outcomes observed after consumption of the corresponding dietary supplement recipes, such as changes in body weight, changes in blood pressure or lipid profiles, changes in sleep quality, changes in activity performance metrics, or self-reported symptom scores or satisfaction scores. Using this training dataset, the gradient-boosted decision tree model may be trained to learn correlations between input features (user data and recipe composition) and output outcomes, such that, for a new user and a candidate recipe, the model can output one or more predicted outcome scores that can be used to score and rank candidate recipes as described above.

By way of a non-limiting example of inputs and outputs, the remote server 102 may receive, for a given user, an input vector comprising: (a) a set of biometric and health/fitness features derived from health/fitness tracker device 104 data (e.g., average daily step count over the last 30 days, average sleep duration, resting heart rate), (b) medical test data 105 features (e.g., most recent hemoglobin A1c level, serum vitamin D concentration, LDL and HDL cholesterol levels), (c) genetic test data 106 features (e.g., the presence or absence of genetic variants associated with slow metabolism, inflammation, or nutrient processing), (d) fitness data 107 (e.g., age, sex, weight, dietary preferences or exclusions, and a selected goal such as “weight loss” or “improved cardiovascular health”), and (e) a representation of a candidate dietary supplement recipe (e.g., a fixed-length vector in which each element encodes the amount, level, or concentration of a corresponding dietary supplement component, such as specific vitamins, minerals, amino acids, or herbal extracts). For training examples, the corresponding output label for each input may comprise, for instance, a scalar or vector-valued score indicating the change in one or more physiological parameters (e.g., change in body weight over 8 weeks, change in blood pressure, or change in sleep quality index) and/or a composite “goal achievement” score derived from these changes. In deployment, the remote server 102 may generate a plurality of candidate dietary supplement recipes for the user, apply the trained model to compute predicted outcome scores for each candidate recipe, and select, recommend, or automatically transmit to the supplement dispenser 103 the recipe whose predicted score satisfies one or more criteria (e.g., maximizes the probability of achieving the user’s selected health/fitness goal, while respecting constraints such as safety limits, allergies, or user-specified exclusions).

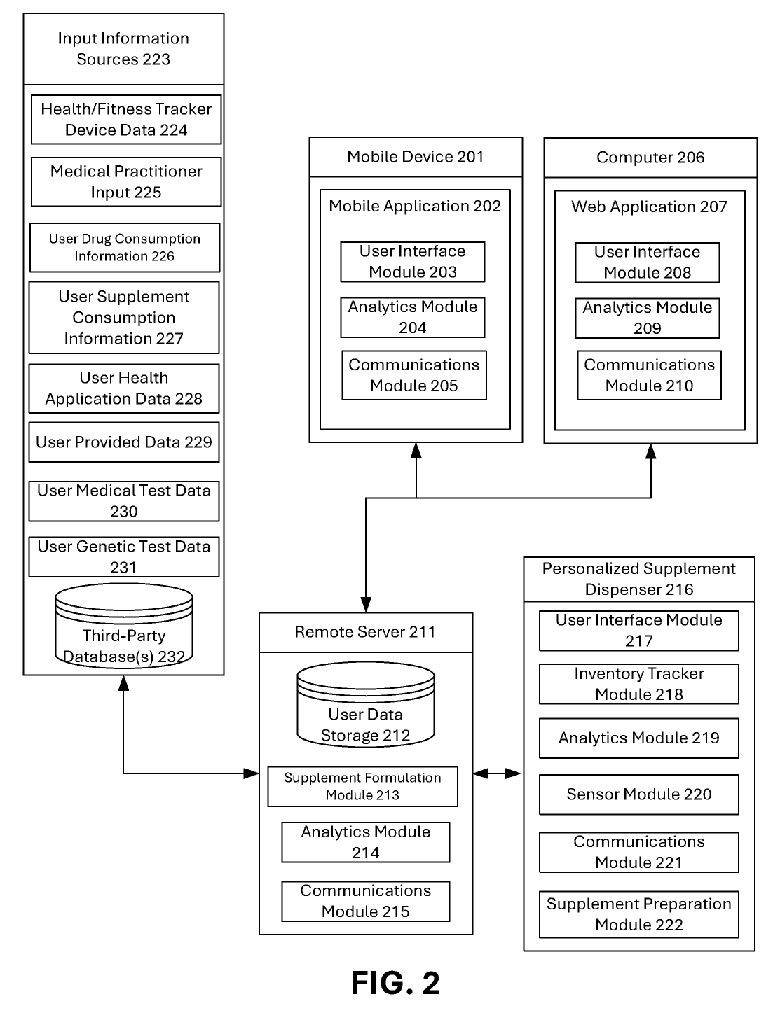

FIG. 2 is a block diagram showing aspects of an exemplary system 200 for generating a personalized dietary supplement according to the present disclosure. This figure highlights potential data sources and the layout of modules used in aspects of the systems described herein.

In this non-limiting example, the system 200 comprises five general components. A remote server 211 is shown (same as remote server 102), which includes a user data storage 212, a supplement formulation module 213, an analytics module 214, and a communications module 215. The user data storage 212 may store, e.g., any of the user health and/or fitness, or other data, described herein. For example, data collected from one or more wearable devices, sensors, or from the user directly or indirectly, may be stored by the user data storage 212. Note that the presence of user data storage 212 as part of the remote server 211 is an optional configuration; in some aspects, the user data storage may comprise a database or other form of storage hosted separately from the remote server 211. The supplement formulation module 213 of the remote server 211 may comprise executable software code for generating new dietary supplement recipes, or modifying existing recipes, using any of the algorithms, statistical techniques, or AI/ML models describes herein. For example, supplement formulation module 213 may be configured to train and/or perform inference operation using any such AI/ML models, or to perform any algorithms described herein. The supplement formulation module 213 may thus be used to create and modify dietary supplement recipes, and to score or rank candidate dietary supplement recipes (e.g., in order to identify a top-scoring recipe that is best-suited for a user based on a user-selected dietary, health, or fitness goal, or based on any other criteria described herein).

The analytics module 214 may comprise executable software code for tracking and/or analyzing user dietary, health, and/or fitness data. For example, the analytics module 214 may be configured to generate charts, graphs, or other text or visual representations based on the user dietary, health, and/or fitness data. For example, the analytics module 214 may track a user’s progress towards a desired health and/or fitness goal, or generate rojections based on the user’s current and/or historical biometric, health, fitness, and/or activity data. The output generated by the analytics module 214 may be provided to the user (or to a third party, such a medical or health professional). The analytics module 214 may also be configured to analyze user data and/or data received from the personalized dietary supplement dispenser 216 (same as supplement dispenser 103) in order to determine or track user preferences, to make recommendations, and/or to automate the ordering of dietary supplement recipe components. For example, the analytics module 214 may analyze the parameters for dietary supplement recipes manually created (or modified) by the user, or the frequency and/or timing of the user generating dietary supplements using the personalized dietary supplement dispenser 216, and generate suggestions for the user regarding new dietary supplement components or recipes to consider. The analytics module 214 may also analyze usage data in order to determine when additional dietary supplement components should be ordered (e.g., due to a low quantity or amount in the personalized dietary supplement dispenser 216), and may be configured to prompt the user to confirm an order to automatically place an order for the required dietary supplement components. The remote server 211 further includes a communications module 215 that may be used to communicate with the personalized dietary supplement dispenser 216, a mobile device 201 (same as mobile device 101), a computer 206 and various input information sources 223. In some aspects, the communications module may be configured to communicate with any combination of these system 200 components, via a wireless or wired connection.

As explained above, a supplement dispenser 216 described herein may be operated or controlled by a mobile device 201, or various other input devices (e.g., a tablet, dedicated controller, laptop, desktop, etc.). In this non-limiting example the remote server 211 is configured to communicate with a mobile device 201 and/or a computer 206. As shown by FIG. 2, each of these devices may comprise an application that includes a user interface module, an analytics module, and a communications module. More specifically, mobile device 201 includes mobile application 202, which further includes user interface module 203, analytics module 204, and communications module 205. Computer 206 includes web application 207, which further includes user interface module 208, analytics odule 209, and communications module 210. Likewise, personalized dietary supplement dispenser 216 includes user interface module 217, analytics module 219, and communications module 221. The analytics module and the communications module, respectively, may perform any or all of the functions of the analytics module 214 and the communications module 215 executed on the remote server 211. The user interface module 203/208, may comprise executable software code for generating a GUI that allows the user to interact with and/or control the personalized dietary supplement dispenser 216. This GUI may, e.g., allow a user to create, select and/or modify dietary supplement recipes, to input dietary, health and/or fitness information, and to set or select health and/or fitness goals or outcomes, etc. In some aspects, the GUI may allow the user to see the current inventory level of the dietary supplement components in the personalized dietary supplement dispenser 216, and/or to order new or additional dietary supplement components for the personal dietary supplement dispenser.

In this example, the remote server 211 is shown in communication with several input information sources 223. A non-limiting set of examples include: health/fitness tracker device data 224, medical practitioner input 225, user drug consumption information 226, user supplement consumption information 227, user health application data 228, user provided data 229, user medical test data 230, user genetic test data 231, and one or more third-party databases 232. Health/fitness tracker device data 224 may comprise, e.g., biometric, physiological, and/or activity data obtained using one or more wearable devices, or sensors integrated into or in communication with, devices used by the user (e.g., smart watches, cell phones, dedicated fitness tracker devices). For example, a user’s smart watch may provide heart rate, blood oxygen saturation, or sleep stage/status data for the user. Medical practitioner input 225 may comprise, e.g., recommended amounts, quantities, concentrations, or volumes of one or more dietary supplement components for the user to consume (daily, or according to any other schedule), or one or more target goals for biometric or physiological parameters, provided by a doctor, nurse, dietician, or other medical/health practitioner (or a laboratory directed by any of the foregoing). For example, medical practitioner input 225 may comprise a recommended daily amount of one or more itamins, probiotics, or other dietary supplement components, or a target weight for the user. User drug consumption information 226, and user supplement consumption information 227, may comprise information about drugs and supplements, respectively, taken by the user. In some cases, a user may set a desired daily minimum or maximum consumption level for a given dietary supplement. Moreover, certain drugs and dietary supplement components may be incompatible or result in adverse effects. Thus, in some aspects it is advantageous for the system 200 to account for other drugs or supplements that a user may consume besides those provided by the personalized dietary supplement dispenser 216. User health application data 228 may comprise any biometric, physiological, health and/or fitness parameters, obtained from a third-party application (e.g., separate from the mobile application 202). For example, a user may utilize a fitness tracker device that collects biometric data from the user and provides that data to a third-party. The remote server 211 may be configured to communicate with the third party and obtain third-party application data. User provided data 229 may comprise any information, data, or parameters provided by a user (e.g., biometric, physiological, health and/or fitness parameters, medical history, target health and/or fitness goals, etc.). Various examples of user provided data 229 are described herein and may be accounted for when the server 211 (or any other component of the system 200) generates, modifies, and/or scores personalized dietary supplements, or as part of any analytics functions described herein. User medical test data 230 may comprise, e.g., biometric or physiological data for the user obtained from one or more tests or diagnostic assays (e.g., a bloodwork panel conducted by a laboratory). Similarly, genetic test data 231 may comprise genetic data concerning the presence of absence of specific biomarkers, or DNA sequencing data, for the user. This figure also shows that the remote server 211 may communicate with one or more third-party databases 232. This includes any third-party hosted or controlled servers, databases, or other sources of user data or information. For example, the remote server 211 may be configured to access a third-party database containing current or historical health and/or fitness data for the user.

The system 200 shown in FIG. 2 also includes a personalized dietary supplement dispenser 216, which may perform any of the functions of a dietary supplement dispenser described herein. In this case, the personalized dietary supplement dispenser 216 includes a user interface module 217, an analytics module 219, and a communications module 221, which may each perform any of the functions provided by the corresponding components in the mobile device 201, the computer 206, and the remote server 211, discussed above. In brief, the user interface module 217 comprises executable software code for generating a GUI that allows a user to control the personalized dietary supplement dispenser 216, the analytics module 219 may analyze dietary supplement consumption data, generate suggestions, and/or recommend or place an order when dietary supplement component levels are low.

As discussed above, in some aspects the systems described herein may track the quantity, amount, concentration, and/or volume of the dietary supplement components stored in the personalized dietary supplement dispenser 216. In this example, the personalized dietary supplement dispenser 216 further comprises an inventory tracker module 218, and a sensor module 220. The sensor module 220 may comprise, e.g., one or more sensors configured to detect and/or measure one or more parameters of the dietary supplement components stored in the personalized dietary supplement dispenser 216 (e.g., one or more scales, each positioned to weight the amount of a different dietary supplement component). In some aspects, the sensor may comprise a mechanical sensor configured to detect a level or volume of a given dietary supplement component, or of a liquid (e.g., water) stored in the personalized dietary supplement dispenser 216. Sensor data collected by the one or more sensors of the sensor module 220 may be used by the inventory tracker module 218 to maintain an inventory of the dietary supplement components available in the personalized dietary supplement dispenser 216. In some aspects, the sensor module 220 may comprise one or more sensors configured to detect machine-readable labels or codes provided on containers that contain the dietary supplement component. The label or code may, e.g., identify the type and/or source, amount, quantity, volume, and/or expiration date of a dietary supplement component. In such cases, the inventory tracker module 218 may utilize data provided by the machine-readable label or code to determine which dietary supplement components are available to the personalized dietary supplement dispenser 216. Moreover, this data may be used by the analytics module 219 (e.g., to track usage, and to provide information such as expiration dates, which are relevant to the determination as to whether to recommend that the user order additional or new dietary supplement components).

The personalized dietary supplement dispenser 216 in this example further includes a supplement preparation module 222. This module comprises executable software code for controlling the physical preparation of dietary supplements by the personalized dietary supplement dispenser 216. For example, this module may be configured to cause the personalized dietary supplement dispenser 216 to combine and/or mix one or more dietary supplement components, and optionally one or more liquids, to produce a personalized dietary supplement for the user based on a recipe received from the remote server 211 or a recipe created or modified by the user (e.g., using the mobile device 201, the computer 206, or via a GUI or physical interface provided by the personalized dietary supplement dispenser 216). The supplement preparation module 222 may communicate with any of the other modules (e.g., the inventor tracker module, the analytics module 219, or the communications module 221).

It is envisioned that the functionality provided each of the modules of the system 200 may, in some aspects, be performed by a single module capable of handling multiple functions. Similarly, any of the modules (or functions thereof) may, in some aspects, be performed by a different component of the system 200. For example, in this case the supplement formulation module is part of the remote server 211. In other aspects, the supplement formulation module may instead be part of the personalized dietary supplement dispenser 216. Furthermore, in some aspects a system for preparing personalized dietary supplements may omit any of the modules (or functionality) shown in this example. For example, a personalized dietary supplement dispenser 216 may be configured to generate personalized dietary supplements for a user based on manual input (e.g., a user creating or modifying recipes stored in the personalized dietary supplement dispenser 216) without the need for a remote server 211. The personalized dietary supplement dispenser 216 may be configured to communicate with the input information sources 223, and any other local or remote sources of data or information, without utilizing the remote server 211 as an intermediary.

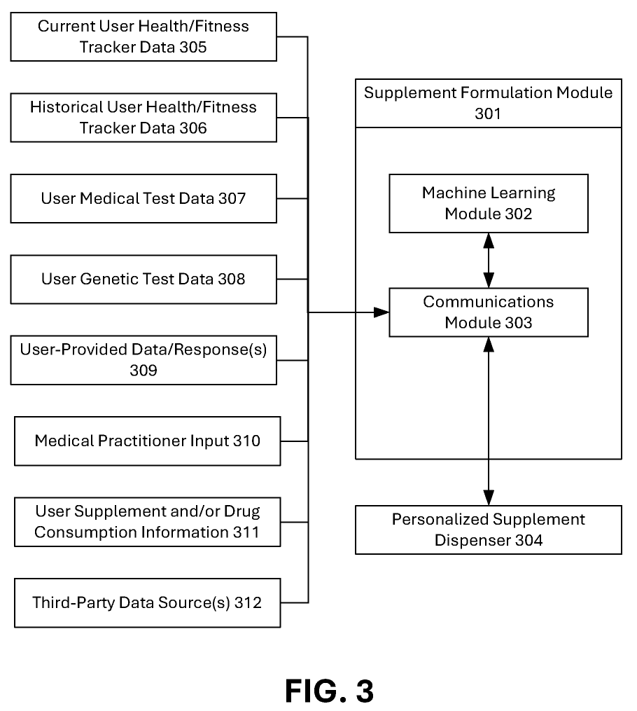

FIG. 3 is a block diagram 300 showing aspects of an exemplary system for generating a personalized dietary supplement according to the present disclosure. This figure highlights potential data sources and the layout of modules used in some aspects of the systems described herein. As discussed above in the context of FIG. 2, a system according to disclosure may utilize various sources of input information (e.g., the sources listed as elements 305-312 in FIG. 3). This information may be obtained from these sources directly or indirectly, and used by a supplement formulation module 301 integrated into a remote server or into a personal dietary supplement dispenser 304. In this example, the information is obtained using a communications module 303, and provided to machine learning module 302. The machine learning module 302 may perform model training, fine-tuning, or inference using this information as discussed above (e.g., to generate personalized dietary supplement recipes or to score candidate recipes based on any parameters described herein).

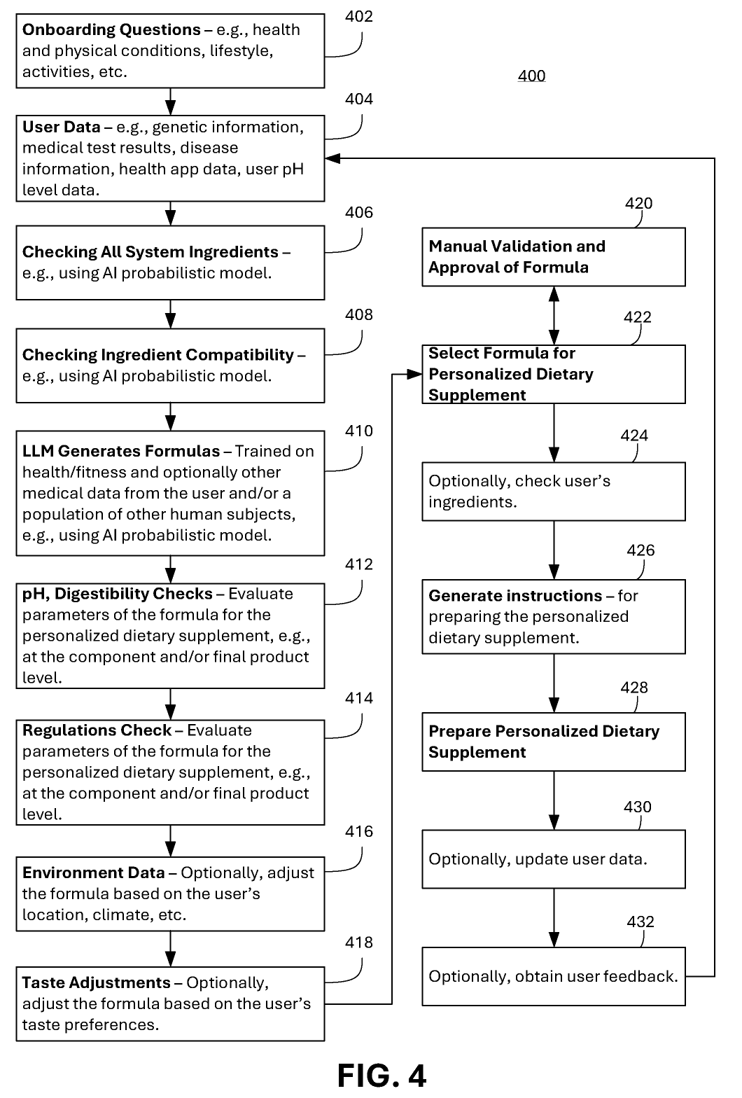

FIG. 4 is a flow chart showing an exemplary method for generating a personalized dietary supplement according to the present disclosure. As illustrated by this figure, an exemplary method for generating a personalized dietary supplement may comprise obtaining, at step 402, input from a user (e.g., via onboarding questions) regarding the user’s health and/or physical conditions, lifestyle, activities, etc. This input may be obtained via any mobile device or computer described herein (e.g., a mobile device 201 or computer 206), or via a remote source (e.g., a remote server 211). Such information may include, e.g., information about the user’s level of fitness, daily activities, inherited or acquired medical conditions or diseases, etc. Optionally, the onboarding questions may collect information about the user’s goals (e.g., weight loss, improved mental wellbeing, improved cardiovascular health, or any other goal described herein). At step 404, user data is obtained from one or more sources (e.g., as described in the context of FIG. 2 or elsewhere herein). Such data may comprise, e.g., genetic information, medical test results, disease nformation, data from one or more health-related applications (e.g., a smartphone app) or biometric sensors/devices (e.g., from a smartwatch, or other wearable device comprising one or more biometric or health-related sensors). As shown by this figure, user data may also include pH level data related to one or more tissues, organs, or body surfaces of the user. At step 406, one or more AI models may be used to check the ingredients (also referred to as “components” herein) available to the system and, at step 408, the compatibility of such ingredients. For example, a pre-trained model may be used to determine whether two or more ingredients stocked by personal dietary supplement dispenser are incompatible, e.g., for health-related reasons, due to taste, texture, or other organoleptic preferences, due to solubility issues, or for any other reason. At step 410, an LLM or other probabilistic AI model is used to generate one or more dietary supplement formulas. This LLM or other probabilistic AI model may be pre-trained on health/fitness and optionally other medical data from the user and/or from a population of other human subjects (e.g., a population of users of a similar age, gender, weight, or any combination thereof). At steps 412 and 414, the generated formulas may be evaluated (e.g., ranked and/or modified) based on one or more parameters (e.g., pH or digestibility) and/or based on compliance with one or more regulations (e.g., target or maximum recommended dosage amounts/levels established by the FDA or any other agency). These evaluations may be performed at the component level and/or at the final product level. For example, the formula as a whole may be evaluated to confirm that the level of a vitamin is below a maximum recommended daily threshold.

Optionally, at steps 416 and 418, the process may further include steps wherein environmental data (e.g., the user’s location, climate, etc.) and taste adjustments are considered (e.g., the user’s taste preferences may be used to rank and/or modify the generated formulas). The LLM or other probabilistic AI model may account for either of these parameters when generating the initial formulas. At step 420, the formula may be manually validated and approved by a user.

At step 422, a formula for the personalized dietary supplement is selected from the set of one or more previously generated formulas. This selection may comprise one or more rounds of ranking and/or modifying formulas. For example, several initial formulas ay be generated, modified and/or ranked based on the user’s environment data and/or taste preferences (as described above), and a final selection may be made based on any combination of criteria described herein. At this point, user input and/or approval may be obtained (e.g., via a GUI of a mobile device application, or on the personal dietary supplement dispenser. This input may validate the automatically generated formula and/or include modifications to the formula. Alternatively, a user may configure a personal dietary supplement dispenser to automatically proceed and generate personalized dietary supplements (e.g., based on a pre-set schedule) without this validation/approval step. When a final recipe is selected (and optionally validated or approved by the user), the following optional step 424 is to check to confirm that the ingredients needed for the selected formula are available (e.g., stocked in the device, available to the user, or orderable from a third party). At step 426, instructions for preparing the selected formula may be generated (e.g., based on one or more LLMs or probabilistic AI models) and, at step 428, the personalized dietary supplement is prepared by the personal dietary supplement dispenser (step 428). Optionally, at step 430, user data may be updated and/or, at step 432, feedback may be collected from the user, as shown by this figure.

Any of the functions, including the generative AI aspects, described in the foregoing example may be performed on or by the personal dietary supplement dispenser, on a communicatively linked device or computer (e.g., a user’s mobile device, computer, or other electronic device), or remotely (e.g., by a server or cloud-based computing platform communicatively linked to the personal dietary supplement dispenser or a device or computer capable of communicating with the personal dietary supplement dispenser. Similarly, user input (e.g., regarding the modification of a recipe, or providing health and/or fitness data) may be collected by any means, including via a GUI of the personalized dietary supplement device, or of a communicatively linked electronic device, computer, server, or other platform, either directly or via a third party (e.g., such data may have been initially obtained from the user via a third-party application).

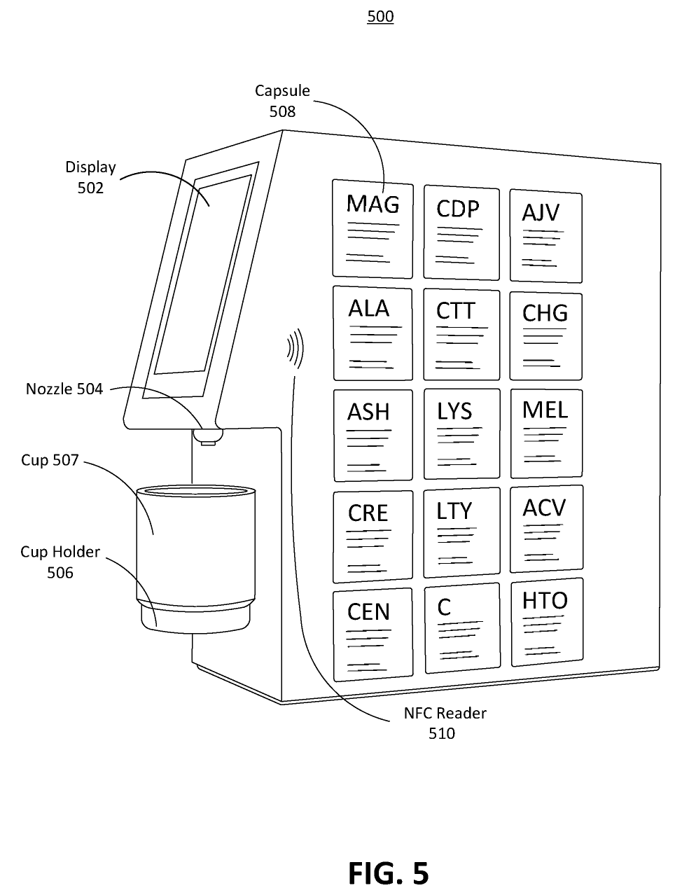

FIG. 5 is a diagram 500 of a personalized dietary supplement dispenser 216. As shown in diagram 500, personalized dietary supplement dispenser 216 may include a plurality of capsules (e.g., capsule 508). Although 15 capsules are shown, it should be noted that fewer capsules may be installed in personalized dietary supplement dispenser 216. Likewise, certain versions of personalized dietary supplement dispenser 216 may accommodate a greater number of capsules. In an exemplary aspect, personalized dietary supplement dispenser 216 is implemented as a device for preparing of a mixture of supplements, in which the plurality of capsules 508 are received via a corresponding plurality of ingredient interfaces, each ingredient interface being configured to be couplable to a respective replaceable container such as capsule 508.

Personalized dietary supplement dispenser 216 further features display 502, that a user may interact with to select a particular supplement recipe for generation. The supplement may be dispensed via nozzle 504 into cup 507 (which may be a user-provided container), which is supported by cup holder 506. In some aspects, personalized dietary supplement dispenser 216 further comprises a structural frame supporting display 502 and defining a front portion of the device at which outlet nozzle 504 is disposed above cup holder 506, which serves as a cup platform for receiving cup 507 during dispensing of a prepared mixture. Personalized dietary supplement dispenser 216 may also have an NFC reader 510 to establish a connection with a mobile device 201. In an exemplary configuration, NFC reader 510 is located at a marked area adjacent a container holder for capsules 508 and is configured to read NFC tags on the capsules, thereby enabling personalized dietary supplement dispenser 216 to transmit information about installed containers to a server, to receive and present server-provided slot assignments for each capsule on display 502, and to receive preparation formulas specifying slot identifiers, pump activation durations, and pump drive voltages.

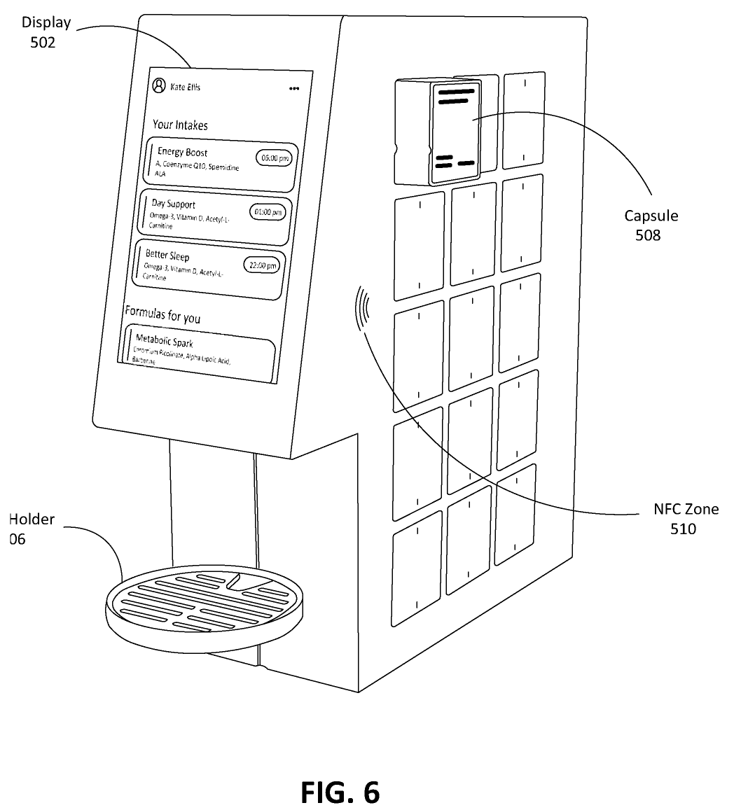

FIG. 6 is a diagram 600 of personalized dietary supplement dispenser 216 with a protruding capsule 508. Personalized dietary supplement dispenser 216 comprises a plurality of ingredient interfaces that can receive the plurality of capsules. As shown in FIG. 6, capsule 508 is protruding out of one of the ingredient interfaces. Each ingredient interface in personalized dietary supplement dispenser 216 is configured as an ingredient interface of the device that is couplable to a replaceable container, such as capsule 508, so that when capsule 508 is fully inserted, the corresponding ingredient interface fluidically couples capsule 508 to a hydraulic system of the device and electrically and logically associates capsule 508 with an ingredient identifier in an ingredient identifier list used by an electronic controller of the device.

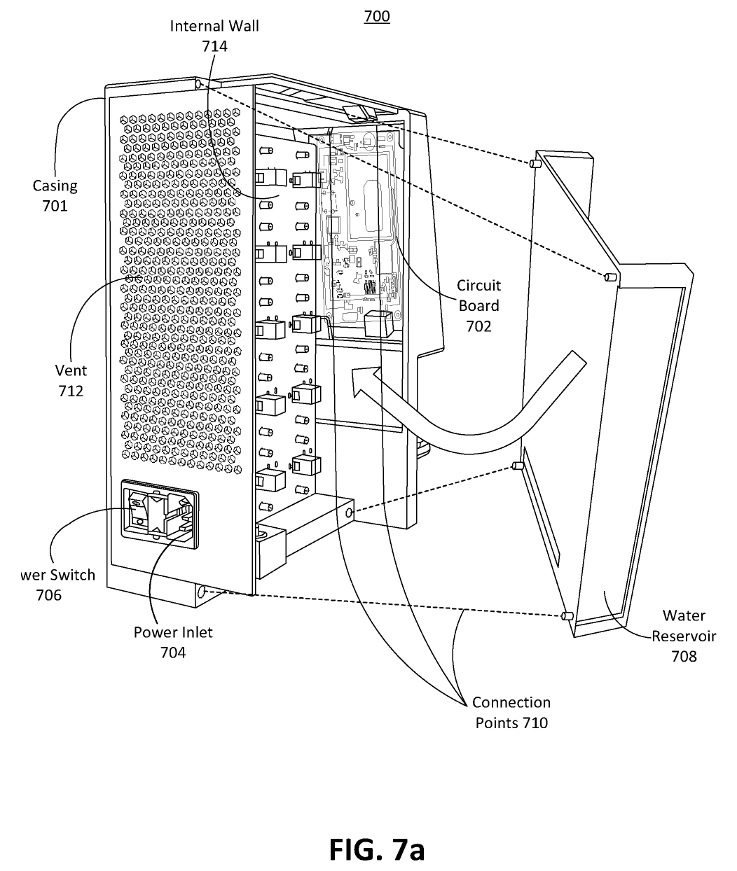

FIG. 7a is a diagram 700 of a set of internal components of personalized dietary supplement dispenser 216. FIG. 7 depicts personalized dietary supplement dispenser 216 from a rear view. The rear side of casing 701 includes vent 712, which allows heat to exit personalized dietary supplement dispenser 216. The rear side of casing 701 further features power inlet 704, which receives a power cable, and power switch 706, which can be toggled by a user to turn personalized dietary supplement dispenser 216 on or off. In some aspects, power inlet 704 is implemented as a rear power inlet, power switch 706 is implemented as a rear on/off switch, and a power cord connects power inlet 704 to a power supply supported by a rear, upper internal bracket within a structural frame of personalized dietary supplement dispenser 216.

Internally, behind display 502, personalized dietary supplement dispenser 216 features circuit board 702, which includes all hardware components for executing user interface module 217, inventory tracker module 218, analytics module 219, sensor module 220, communications module 221, supplement preparation module 222, and/or supplement formulation module 301. In an exemplary configuration, circuit board 702 is a printed circuit board mounted within a central space of the structural frame of personalized dietary supplement dispenser 216 and connected at an upper portion to the power supply supported by the rear, upper internal bracket, and circuit board 702 further supports a real-time operating system (RTOS) controller configured to control operation of metering pumps of the hydraulic system and to implement a rinse function, as well as a front display controller in communication with the RTOS controller and configured to issue preparation commands. In such aspects, the metering pumps are electrically connected to circuit board 702 by electrical connectors so that the RTOS controller can actuate selected metering pumps based on preparation instructions derived from general recipes.

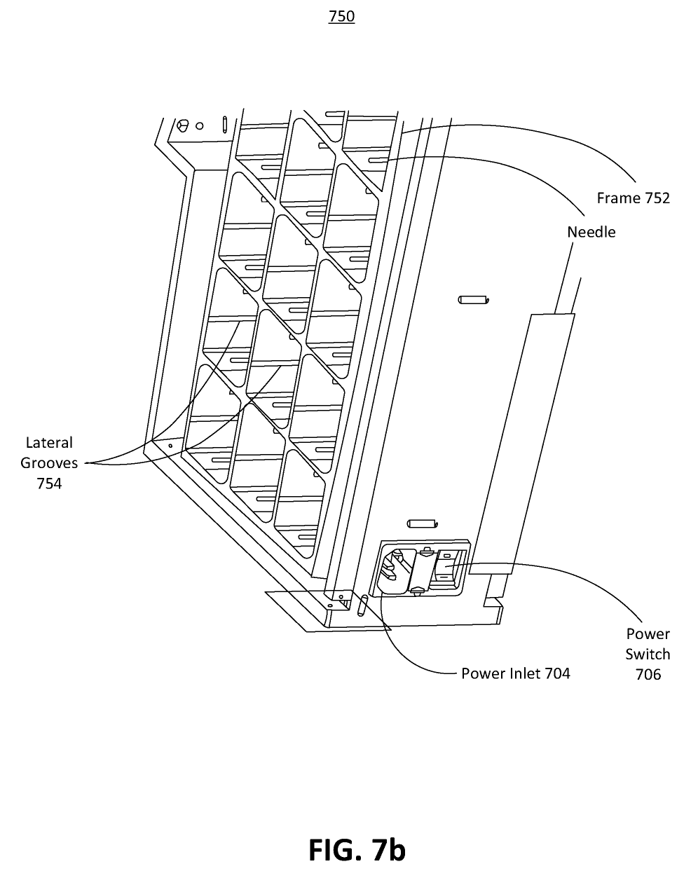

The exploded view of FIG. 7 further shows water reservoir 708, which is fixed in personalized dietary supplement dispenser 216 via connection points 710. Water reservoir 708 may have a height and width that generally matches the height and width of personalized dietary supplement dispenser 216 for easy integration. It should be noted that diagram 700 does not show the piping and hydraulic system (shown in FIG. 8) that is attached to internal wall 714. Internal wall 714 masks the capsules described in FIG. 6. In some aspects, water reservoir 708 is implemented as a side-mounted reservoir with a lid that is installable and removable by sliding sideways relative to personalized dietary supplement dispenser 216 or by pulling water reservoir 708 upward, and water reservoir 708 may alternatively be implemented as a boiler for heating water supplied to the hydraulic system of the device.

FIG. 7b is a diagram 750 of a frame that supports a plurality of capsules in personalized dietary supplement dispenser 216. Frame 752 is fixed on an opposite side of internal wall. In an exemplary aspect, each hollow portion of frame 752 receives a capsule. Each hollow portion further includes two lateral grooves 754 that protrude outward. As will be discussed later, each capsule has two lateral grooves that correspond to the lateral grooves of the hollow portion to enable interlocking. This enables the capsule to pass through the hollow portion in the correct orientation because an incorrect orientation will prevent the respective grooves from aligning. In this manner, frame 752 defines, at a cell level, orientation features including lateral grooves 754 in a lower third of each cell that key container insertion in only one orientation relative to internal needles of the ingredient interface, so that each capsule is constrained to an orientation that aligns internal valves of the capsule with the needles of the hydraulic system.

FIG. 8 is a diagram 800 of the piping and hydraulic system of personalized dietary supplement dispenser 216. As shown in diagram 800, a common conduit 802 travels up the internal wall 714 in an alternating manner. In an exemplary aspect, common conduit 802 is formed by tubing that extends from a water valve associated with water reservoir 708 to outlet nozzle 504 disposed at the front portion of personalized dietary supplement dispenser 216 above cup holder 506, and a plurality of branch lines extend from respective metering pumps to connect to common conduit 802 so that liquids can be pumped and mixed for preparation of a supplement mixture. Tubing could be manufactured from several tubes or could be realized by silicon forming.

For each ingredient interface that can receive a capsule, there is a local pump 804 and branch tubing 806 that extends from the common conduit 802 into internal wall 714. In some aspects, each local pump 804 is implemented as a metering pump of the hydraulic system, and a one-way check valve is disposed downstream of each metering pump 804 along the corresponding branch tubing 806 to prevent backflow toward the respective metering pump, with additional one-way valves provided in common conduit 802 upstream of each junction where a branch line 806 connects so as to prevent reverse flow into other branch lines. In operation, an electronic controller of personalized dietary supplement dispenser 216 actuates selected metering pumps 804 based on an instruction to prepare a mixture according to a general recipe, so as to meter ingredient volumes from capsules 508 through branch tubing 806 into common conduit 802, in some cases according to a dosing scheme that specifies a time of actuation of at least one metering pump 804 based on physical properties such as viscosity of the corresponding ingredient.

A rinse pump 810 is connected to water reservoir 708. An upstream valve 808 connected to common conduit 802 is installed to prevent ingredients from moving backwards in common conduit 802. As water travels within common conduit 802 and different ingredients are mixed by individual pumps, a target supplement is generated and output via nozzle 504. In some aspects, rinse pump 810 is a metering pump of the hydraulic system dedicated to rinsing, and, under control of the electronic controller, rinse pump 810 is actuated after completion of ingredient metering to initiate a rinsing flow from water reservoir 708 through common conduit 802 toward nozzle 504, thereby flushing residual ingredients toward outlet nozzle 504 and contributing to hygiene of the dispensing pathway. In other aspects, the hydraulic system is further configured to execute a cleaning cycle using a special cleaning capsule installed in one of the ingredient interfaces, during which the electronic controller actuates pumps 804 and rinse pump 810 to flush and sanitize common conduit 802 and outlet nozzle 504 with a cleaning solution supplied from the special leaning capsule and/or water reservoir 708.

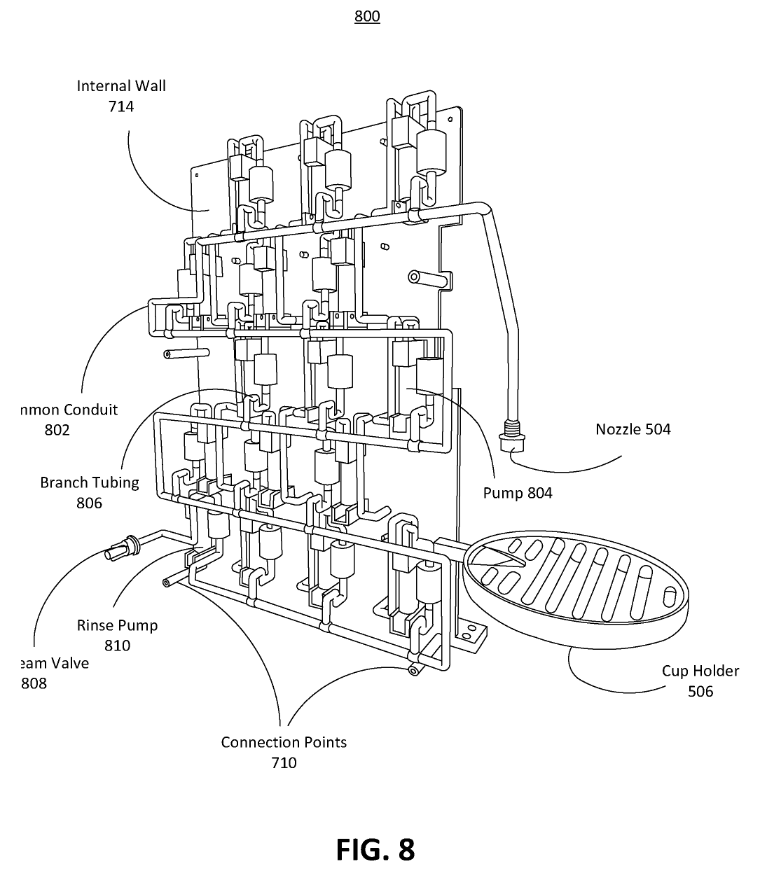

FIG. 9 is a diagram 900 of piping in a branch of the hydraulic system. Diagram 900 shows pipe 904, which receives ingredients from a capsule. Air inlet 902 is present for receiving air. The ingredients are drawn from pump 804 and sent through branch piping, which further includes check valve 906. The drawn ingredients then enter common conduit 802. Check valve 906 ensures that fluids in common conduit 802 to do reverse back into a capsule. In some aspects, pump 804 is fluidically connected to pipe 904 so that, when actuated by the electronic controller, pump 804 extracts liquid ingredients from a coupled capsule through pipe 904 while air inlet 902 admits compensating air into the capsule, the respective flows being regulated such that the liquid and air paths remain separated by check valve 906 and by associated one-way valves in the branch piping.

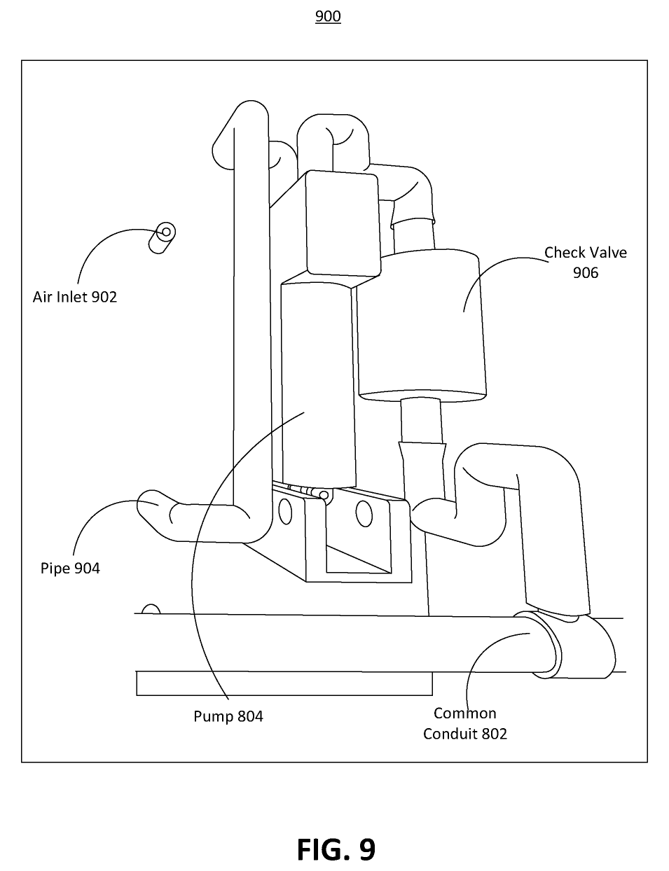

FIG. 10 is a diagram 1000 depicting a plurality of ingredient interfaces. Relative to diagram 800, diagram 1000 depicts the opposite side of internal wall 714. Each ingredient interface includes a capsule holder cap 1002, which features lateral grooves 1004 that are indicative of orientation. Capsule holder cap 1002, together with frame 752 and internal wall 714, forms a plastic grid that serves as a container holder, with each hollow portion defining a respective cell into which a capsule 508 can be inserted in only one orientation dictated by lateral grooves 1004 that correspond to lateral grooves 754 of frame 752 and lateral grooves 1110 of capsule 508.

In an exemplary aspect, needles extend from the hydraulic system into each capsule. For example, needle 1006 is connected to air inlet 902 and needle 1008 is connected to pipe 904. Needle 1006 passes through needle hole 1012 of the ingredient interface and needle 1008 passes through needle hole 1010. In particular, each ingredient interface of the plurality of ingredient interfaces comprises a pair of piercing needles including air needle 1006 and liquid needle 1008 that are fixed relative to the plastic grid defined by capsule holder cap 1002 and frame 752, and that project into a respective cell so as to engage internal valve structures of an installed capsule 508. In such aspects, needle 1008 is fluidically coupled via pipe 904 to a corresponding metering pump 804 to extract liquid from capsule 508, and needle 1006 is fluidically coupled via air inlet 902 to an air path configured to admit air into capsule 508, with the two needles being positioned such that, upon insertion of capsule 508, needle 1008 engages a liquid outlet valve within capsule 508 and needle 1006 engages an air inlet valve within capsule 508. In some implementations, each ingredient interface further comprises a push-to-open cover associated with capsule holder cap 1002 and biased by an internal pusher to keep the corresponding cell closed when no capsule 508 is installed, thereby protecting needles 1006 and 1008 from exposure and accidental contact, and the push-to-open cover is arranged to open upon being pressed by a user for capsule insertion.

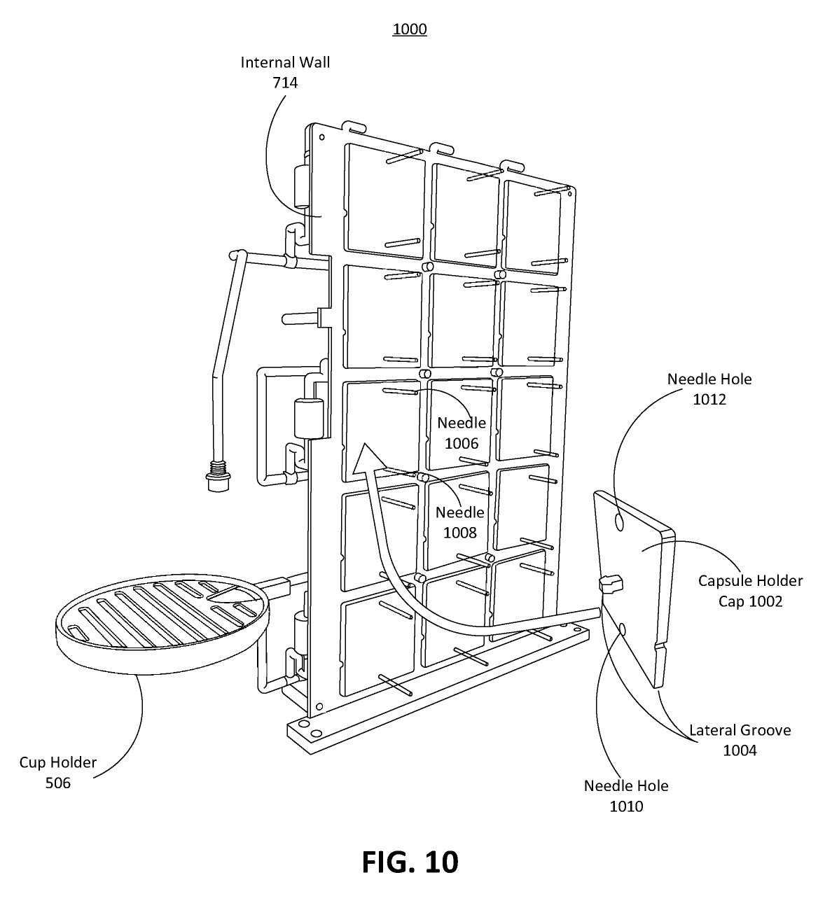

FIG. 11 is a diagram 1100 of an exemplary capsule 508. Capsule 508 may be a rectangular prism with capsule cover 1102 through which ingredients are placed into capsule 508. Needle receiver 1104 receives needle 1006 and needle receiver 1106 receives needle 1008. In some aspects, needle receiver 1106 may be connected to a capsule valve 1108 that enables and/or prevents ingredient flow from capsule 508. In an exemplary embodiment, capsule 508 is implemented as a factory-sealed, single-use container having a generally parallelepiped body with capsule cover 1102 serving as a lid, an affixed NFC tag storing container identification and ingredient data, and two internal one-way valves disposed on an inner surface respectively aligned with needle receivers 1104 and 1106 so as to be engaged and opened by needles 1006 and 1008 when capsule 508 is installed in the corresponding ingredient interface.

Capsule 508 may include lateral grooves 1110 so that insertion and connection to an ingredient interface is in the correct orientation. For example, the frame that receives and holds capsule 508 may have grooves located and shaped to receive the lateral grooves 1110. These lateral grooves 1110 cooperate with lateral grooves 754 of frame 752 and lateral grooves 1004 of capsule holder cap 1002 to realize the cell-level orientation features of each ingredient interface, ensuring that capsule 508 can be inserted only in an orientation in which needle receivers 1104 and 1106 properly align with needles 1006 and 1008 for reliable fluidic engagement.

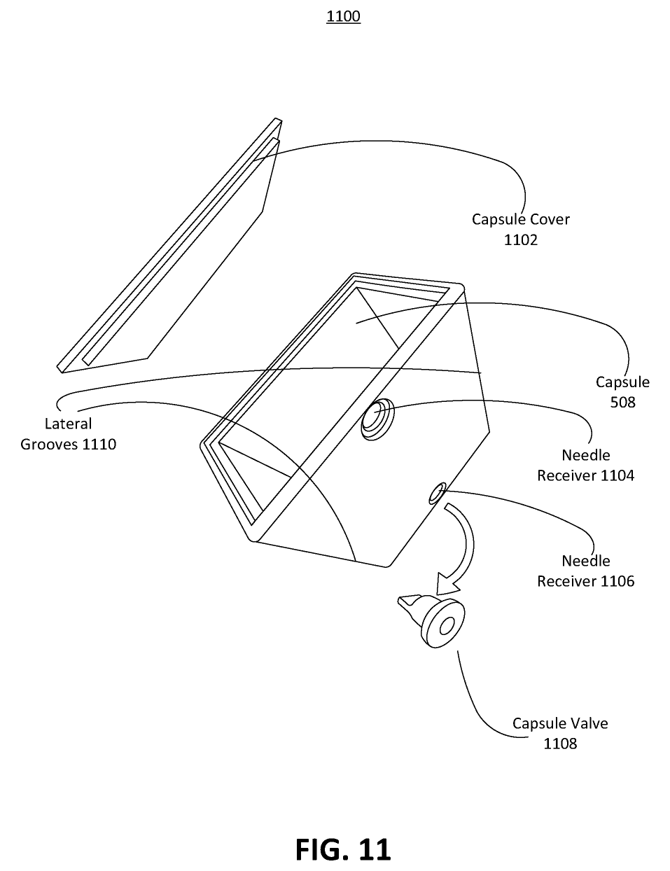

FIG. 12 is a diagram 1200 that shows liquid flow within personalized dietary supplement dispenser 216. As shown in diagram 1200, water is pumped upwards from the water reservoir 708 into common conduit 802. Local branches that supply ingredients may be sequentially actuated based on the formula selected by the user. As water rises through common conduit 802, the local pumps (e.g., pump 804) push ingredients at the required amount into common conduit 802 for mixture. The total mixture is then released via nozzle 504.

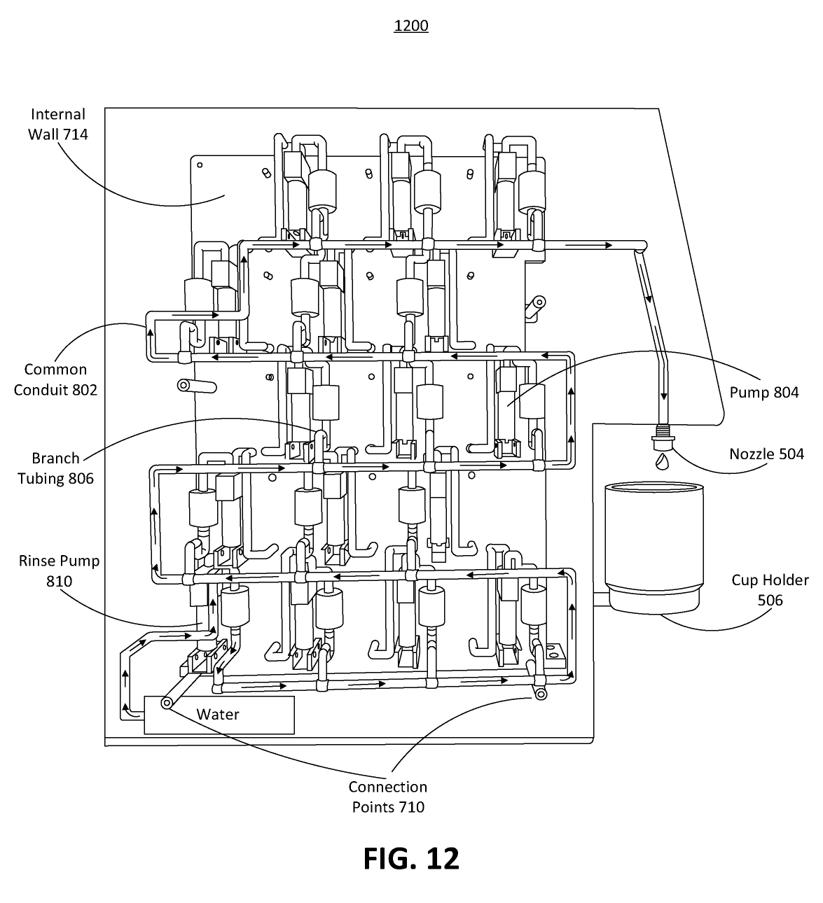

FIG. 13 is a diagram 1300 that shows a first user interface output on a displayof personalized dietary supplement dispenser 216. In particular, the first user interface output features a QR code that a user may scan using mobile device 201 to initiate a pairing process. A user may use a camera of mobile device 201 to scan the QR code or manually enter a pairing code to establish a connection between personalized dietary supplement dispenser 216 and mobile device 201. In some aspects, personalized dietary supplement dispenser 216 further comprises a wireless network interface configured to connect to aWi Fi network, and display 502 is configured to present the QR code shown in diagram 1300 to facilitate pairing of the device with a user profile via a mobile application executed on mobile device 201.

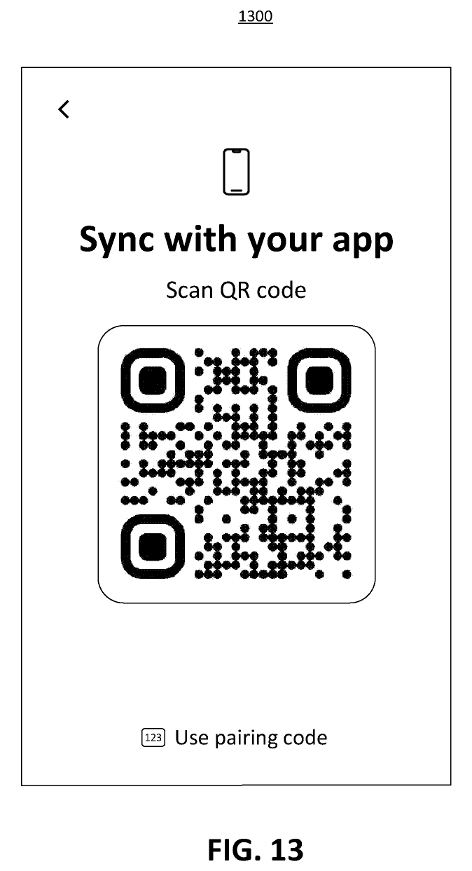

FIG. 14 is a diagram 1400 that shows a second user interface output on a display of personalized dietary supplement dispenser 216. The second user interface output is a menu with the options "capsule status," "add capsules," and "settings." This menu may be generated after pairing is complete with mobile device 201 and the user has logged into their profile on personalized dietary supplement dispenser 216. In operation, selection of the "add capsules" or "capsule status" options causes the electronic controller, via NFC reader 510 located adjacent the capsule holder, to read NFC tags affixed to capsules 508, transmit information about installed capsules 508 to a remote server, receive and display a server provided slot assignment for each capsule, and receive preparation formulas specifying slot identifiers, pump activation durations, and pump drive voltages, thereby integrating capsule registration and recipe execution with the user-specific profile associated with the paired mobile application.

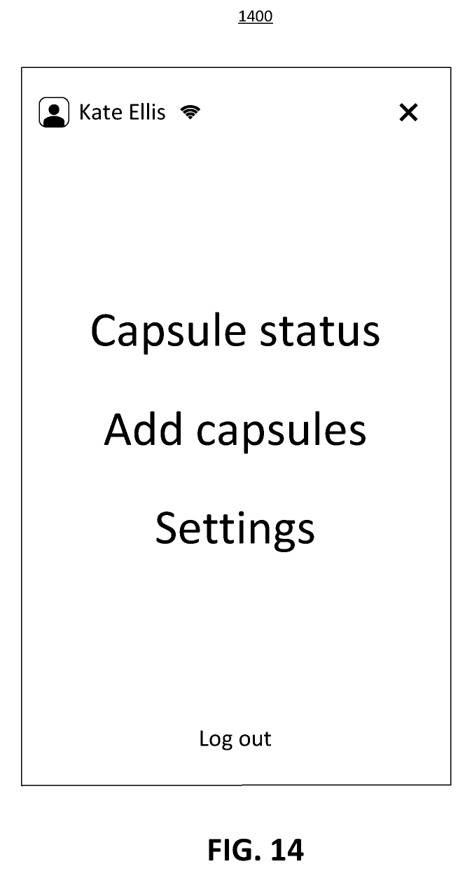

FIG. 15 is a diagram 1500 that shows a third user interface output on a display of personalized dietary supplement dispenser 216. The third user interface output is a menu of formulas that the user can generate based on their preferences and available ingredients. For example, one formula option is titled "metabolic spark," which has the ingredients: chromium picolinate, alpha lipoic acid, and berberine. Another option is titled "brain performance," which features the ingredients: citicoline, rhodiola rosea, acetyl-L-cartinine. A user may select a formula to access more information about the formula or to have personalized dietary supplement dispenser 216 dispense a supplement based on the formula. When a user selects a formula as shown in diagram 1500, the electronic controller interprets the selected formula as a general recipe and, based on installed-container data including the ingredient identifier list and associated properties of ingredients currently available in capsules 508, generates a preparation instruction that specifies, for each ingredient of the recipe, a slot identifier corresponding to a capsule position, a pump activation duration for the corresponding metering pump 804, and a pump drive voltage, so that execution of the preparation instruction produces the desired mixture.

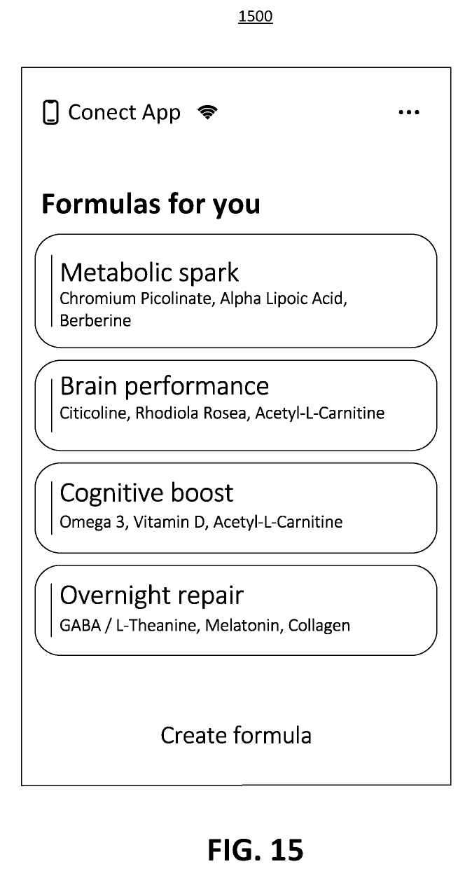

FIG. 16 is a diagram 1600 that shows a fourth user interface output on a display of personalized dietary supplement dispenser 216. The fourth user interface output features a visualization that showcases the attributes a selected formula targets in the human body. For example, a supplement generated based on the formula "energy boost" is shown to target digestion, ectodermal, cardio, and sleep and recovery attributes. The fourth user interface output further lists the ingredients in the formula and the amount of an ingredient used. In response to user confirmation on the interface shown in diagram 1600, the electronic controller treats the confirmation as the instruction to prepare a mixture based on the displayed general recipe and initiates the process of detecting installed capsules 508, updating the ingredient identifier list and remaining-volume data for each capsule, and scheduling actuation of metering pumps 804 and rinse pump 810 to implement the formula.

The fourth user interface output further includes an option to "prepare" a supplement based on the formula.

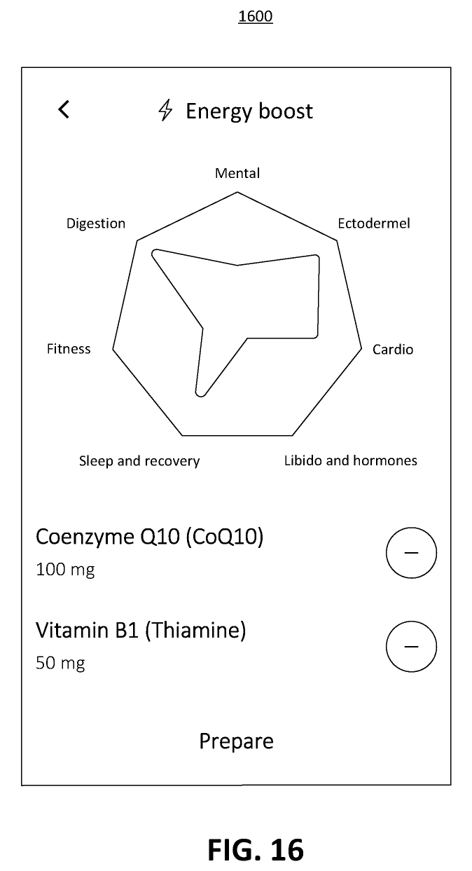

FIG. 17 is a diagram 1700 that shows a fifth user interface output on a display of personalized dietary supplement dispenser 216. The fifth user interface output is generated in response to receiving a user selection of the "prepare" option shown in the fourth user interface output. As shown in diagram 1700, a progress bar is generated that shows how much of the supplement generation is complete. In some aspects, the progress bar may further be accompanied by a duration until completion (e.g., 20 seconds until complete) on the user interface. While the progress bar shown in diagram 1700 advances, the electronic controller actuates selected metering pumps 804 in accordance with the previously generated preparation instruction, including any dosing-scheme-based timing adjustments that account for physical properties such as viscosity of individual ingredients, and subsequently actuates rinse pump 810 to cause a rinsing flow from water reservoir 708 through common conduit 802 toward nozzle 504, so that the user receives a freshly prepared mixture and the hydraulic system is partially cleansed for subsequent operations.

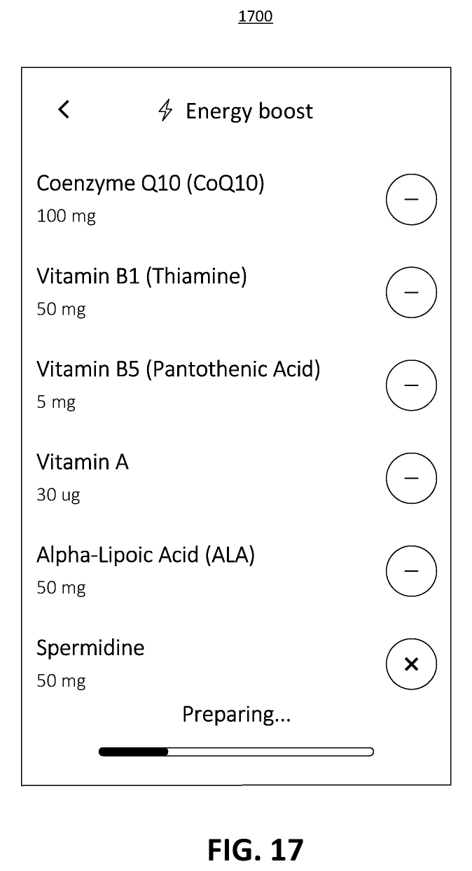

FIG. 18 is a diagram 1800 that shows a sixth user interface output on a display of personalized dietary supplement dispenser 216. The sixth user interface output depicts an indicator of each capsule that is installed in personalized dietary supplement dispenser 216 at a given time. Each indicator may be include a name of the ingredient, an abbreviation of the name, and a status bar that indicates the amount of ingredient remaining in a capsule. For example, in diagram 1800, all ingredients have an equal amount remaining in each capsule. In some aspects, the indicators in diagram 1800 are generated using the ingredient identifier list and associated properties maintained by the electronic controller, including the current remaining volume for each ingredient in each capsule 508, and this information is updated after each dispensing operation based on measured or computed pump activation durations and drive voltages used for metering ingredients from the respective capsules.

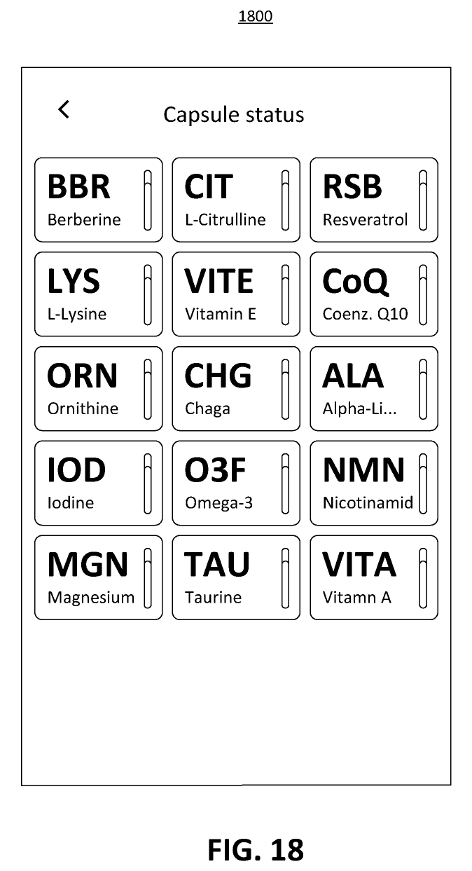

FIG. 19 is a flow diagram of a method 1900 for preparing of a mixture of supplements using a dispensing device.

At 1902, electronic controller (e.g., part of circuit board 702) of the dispensing device (e.g., personalized dietary supplement dispenser 216) receives an instruction (e.g., shown in FIG. 16) to prepare a mixture based on a general recipe.

At 1904, the electronic controller detects one or more replaceable containers (e.g., capsules) currently coupled to at least one of a plurality of ingredient interfaces.

At 1906, the electronic controller determines an ingredient identifier list associated with the one or more replaceable containers and associated properties of ingredients in the ingredient identifier list.

At 1908, in response to the instruction, the electronic controller actuates selected metering pumps (e.g., pump 804) of a hydraulic system of the dispensing device to deliver ingredient volumes from the one or more replaceable containers in accordance with the general recipe and initiate a rinsing flow (e.g., from rinse pump 810) through the hydraulic system toward an outlet (e.g., nozzle 504) of the dispensing device.

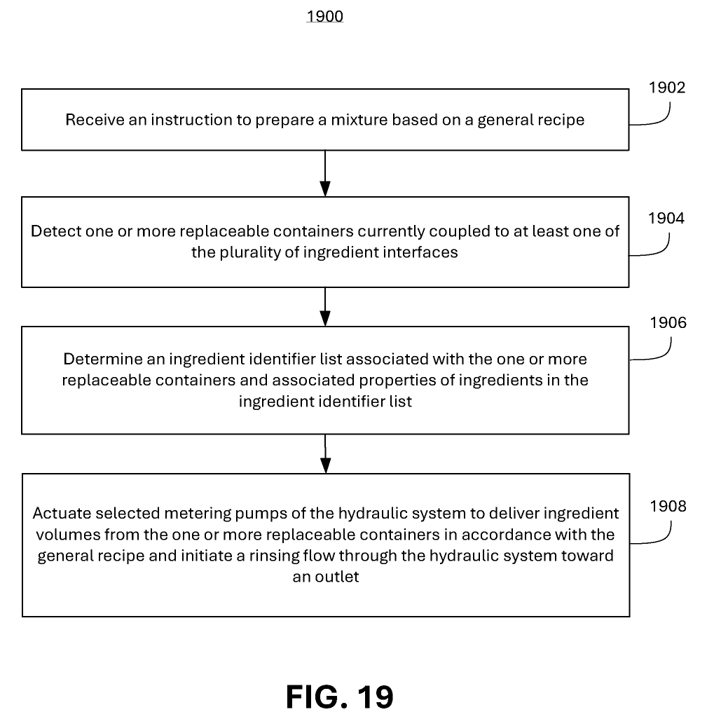

FIG. 20 is a flow diagram of a method 2000 for preparing and delivering a supplement mixture using a dispensing device.

At 2002, a device (e.g., remote server 211, personalized dietary supplement dispenser 216, and/or mobile device 201) identifies user-specific intake criteria based on a user profile. In some aspects, the user profile is linked to the dispensing device by processing, with a mobile application associated with the user profile and executed on mobile device 201, a QR code that is displayed on personalized dietary supplement dispenser 216, there by associating the user profile with that particular dispensing device for subsequent preparation and delivery of supplement mixtures.

At 2004, the device generates a general recipe for supplement intake based on the user-specific intake criteria. In an exemplary configuration, the general recipe is generated in accordance with supplement compatibility, intake recommendations, and periodicity, such that the device takes into account compatibility constraints among supplement ingredients, recommended intake levels, and dosing frequency when formulating the general recipe for the user.

At 2006, personalized dietary supplement dispenser 216 detects one or more replaceable containers (e.g., capsules) currently coupled with at least one of a plurality of ingredient interfaces of the dispensing device. In some aspects, detecting the one or more replaceable containers further comprises registering the one or more replaceable containers by detecting, using an NFC reader of personalized dietary supplement dispenser 216, each container of the one or more replaceable containers, verifying container data for each detected container with remote server 211 and receiving from the server a corresponding slot assignment for installation, transmitting a post-installation confirmation of each container back to the server after the container is installed in its assigned slot, and updating, as doses are dispensed, a remaining volume of liquid in each installed container so that current container status is maintained as part of the device's operating data.

At 2008, personalized dietary supplement dispenser 216 generates a preparation instruction based on the general recipe and installed-container data about the one or more replaceable containers. In some implementations, the installed-container data comprises an ingredient identifier list of the supplement ingredients available in the detected containers together with associated properties of those supplement ingredients, including one or more of physical properties comprising at least viscosity, chemical properties, and a current remaining volume for each ingredient. The preparation instruction generated at 2008 may indicate, for each respective ingredient of the general recipe, a slot identifier corresponding to a physical position of a container within personalized dietary supplement dispenser 216, a pump activation duration for a metering pump associated with that slot, and a pump drive voltage for energizing the metering pump during execution of the recipe. In some aspects, at least one pump activation duration specified in the preparation instruction is set or subsequently adjusted based on physical properties of the respective ingredient, such as its viscosity, so as to ensure that a desired ingredient volume is delivered despite variations in flow behavior.

At 2010, in response to receiving a preparation command via a device interface or a mobile application associated with the dispensing device, personalized dietary supplement dispenser 216 executes the preparation instruction to generate a mixture by actuating selected metering pumps to meter ingredient volumes from the one or more replaceable containers into a fluid path based on the general recipe and subsequently initiating a rinsing flow through the fluid path.

At 2012, personalized dietary supplement dispenser 216 dispenses the mixture to a receptacle for user consumption.

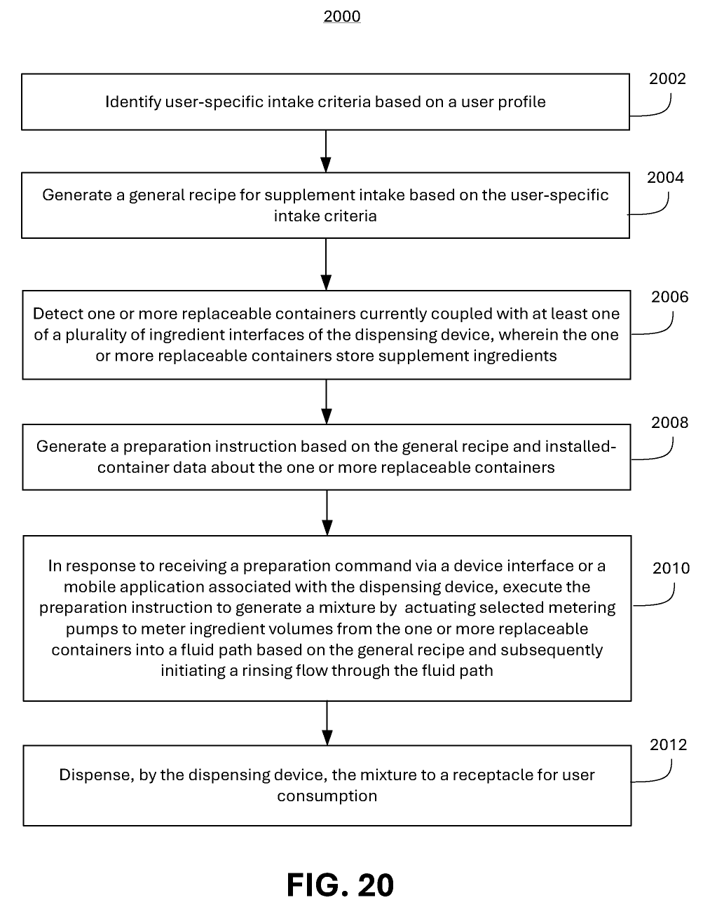

FIG. 21 is a block diagram illustrating a computer system 20 on which aspects of systems and methods for preparing a mixture of supplements using dispensing device may be implemented in accordance with an exemplary aspect. The computer system 20 can be in the form of multiple computing devices, or in the form of a single computing device, for example, a desktop computer, a notebook computer, a laptop computer, a mobile computing device, a smart phone, a tablet computer, a server, a mainframe, an embedded device, and other forms of computing devices.

As shown, the computer system 20 includes a central processing unit (CPU) 21, a system memory 22, and a system bus 23 connecting the various system components, including the memory associated with the central processing unit 21. The system bus 23 may comprise a bus memory or bus memory controller, a peripheral bus, and a local bus that is able to interact with any other bus architecture. Examples of the buses may include PCI, ISA, PCI-Express, HyperTransport, InfiniBand™, Serial ATA, I²C, and other suitable interconnects. The central processing unit 21 (also referred to as a processor) can include a single or multiple sets of processors having single or multiple cores. The processor 21 may execute one or more computer-executable code implementing the techniques of the present disclosure. For example, any of the software commands/steps discussed in FIGS. 1-20 may be performed by processor 21 (part of the circuit board of the dispensing device, an electronic controller, or a server communicating with the dispensing device). The system memory 22 maybe any memory for storing data used herein and/or computer programs that are executable by the processor 21. The system memory 22 may include volatile memory such as a random access memory (RAM) 25 and non-volatile memory such as a read only memory (ROM) 24, flash memory, etc., or any combination thereof. The basic input/output system (BIOS) 26 may store the basic procedures for transfer of information between elements of the computer system 20, such as those at the time of loading the operating system with the use of the ROM 24.

The computer system 20 may include one or more storage devices such as one or more removable storage devices 27, one or more non-removable storage devices 28, or a combination thereof. The one or more removable storage devices 27 and non-removable storage devices 28 are connected to the system bus 23 via a storage interface 32. In an aspect, the storage devices and the corresponding computer-readable storage media are power-independent modules for the storage of computer instructions, data structures, program modules, and other data of the computer system 20. The system memory 22, removable storage devices 27, and non-removable storage devices 28 may use a variety of computer-readable storage media. Examples of computer-readable storage media include machine memory such as cache, SRAM, DRAM, zero capacitor RAM, twin transistor RAM, eDRAM, EDO RAM, DDR RAM, EEPROM, NRAM, RRAM, SONOS, PRAM; flash memory or other memory technology such as in solid state drives (SSDs) or flash drives; magnetic cassettes, magnetic tape, and magnetic disk storage such as in hard disk drives or floppy disks; optical storage such as in compact disks (CD-ROM) or digital versatile disks (DVDs); and any other medium which may be used to store the desired data and which can be accessed by the computer system 20.

The system memory 22, removable storage devices 27, and non-removable storage devices 28 of the computer system 20 may be used to store an operating system 35, additional program applications 37, other program modules 38, and program data 39. The computer system 20 may include a peripheral interface 46 for communicating data from input devices 40, such as a keyboard, mouse, stylus, game controller, voice input device, touch input device, or other peripheral devices, such as a printer or scanner via one or more I/O ports, such as a serial port, a parallel port, a universal serial bus (USB), or other peripheral interface. A display device 47 such as one or more monitors, projectors, or integrated display, may also be connected to the system bus 23 across an output interface 48, such as a video adapter. In addition to the display devices 47, the computer system 20 may be equipped with other peripheral output devices (not shown), such as loudspeakers and other audiovisual devices.

The computer system 20 may operate in a network environment, using a network connection to one or more remote computers 49. The remote computer (or computers) 49 may be local computer workstations or servers comprising most or all of the aforementioned elements in describing the nature of a computer system 20. Other devices may also be present in the computer network, such as, but not limited to, routers, network stations, peer devices or other network nodes. The computer system 20 may include one or more network interfaces 51 or network adapters for communicating with the remote computers 49 via one or more networks such as a local-area computer network (LAN) 50, a wide-area computer network (WAN), an intranet, and the Internet. Examples of the network interface 51 may include an Ethernet interface, a Frame Relay interface, SONET interface, and wireless interfaces.

Aspects of the present disclosure may be a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store program code in the form of instructions or data structures that can be accessed by a processor of a computing device, such as the computing system 20. The computer readable storage medium may be an electronic storage device, a magnetic storage device, an optical storage device, an electromagnetic storage device, a semiconductor storage device, or any suitable combination thereof. By way of example, such computer-readable storage medium can comprise a random access memory(RAM), a read-only memory (ROM), EEPROM, a portable compact disc read-only memory (CD-ROM), a digital versatile disk (DVD), flash memory, a hard disk, a portable computer diskette, a memory stick, a floppy disk, or even a mechanically encoded device such as punch-cards or raised structures in a groove having instructions recorded thereon. As used herein, a computer readable storage medium is not to be construed as being transitory signals per se, such as radio waves or other freely propagating electromagnetic waves, electromagnetic waves propagating through a waveguide or transmission media, or electrical signals transmitted through a wire.

Computer readable program instructions described herein can be downloaded to respective computing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network interface in each computing device receives computer readable program instructions from the network and forwards the computer readable program instructions for storage in a computer readable storage medium within the respective computing device.

Computer readable program instructions for carrying out operations of the present disclosure may be assembly instructions, instruction-set-architecture (ISA) instructions, machine instructions, machine dependent instructions, microcode, firmware instructions, state-setting data, or either source code or object code written in any combination of one or more programming languages, including an object oriented programming language, and conventional procedural programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a LAN or WAN, or the connection may be made to an external computer (for example, through the Internet). In some aspects, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

In various aspects, the systems and methods described in the present disclosure can be addressed in terms of modules. The term "module" as used herein refers to a real-world device, component, or arrangement of components implemented using hardware, such as by an application specific integrated circuit (ASIC) or FPGA, for example, or as a combination of hardware and software, such as by a microprocessor system and a set of instructions to implement the module's functionality, which (while being executed) transform the microprocessor system into a special-purpose device. A module may also be implemented as a combination of the two, with certain functions facilitated by hardware alone, and other functions facilitated by a combination of hardware and software. In certain implementations, at least a portion, and in some cases, all, of a module may be executed on the processor of a computer system. Accordingly, each module may be realized in a variety of suitable configurations, and should not be limited to any particular implementation exemplified herein.

In the interest of clarity, not all of the routine features of the aspects are disclosed herein. It would be appreciated that in the development of any actual implementation of the present disclosure, numerous implementation-specific decisions must be made in order to achieve the developer's specific goals, and these specific goals will vary for different implementations and different developers. It is understood that such a development effort might be complex and time-consuming, but would nevertheless be a routine undertaking of engineering for those of ordinary skill in the art, having the benefit of this disclosure.

Furthermore, it is to be understood that the phraseology or terminology used herein is for the purpose of description and not of restriction, such that the terminology or phraseology of the present specification is to be interpreted by the skilled in the art in light of the teachings and guidance presented herein, in combination with the knowledge of those skilled in the relevant art(s). Moreover, it is not intended for any term in the specification or claims to be ascribed an uncommon or special meaning unless explicitly set forth as such.

The various aspects disclosed herein encompass present and future known equivalents to the known modules referred to herein by way of illustration. Moreover, while aspects and applications have been shown and described, it would be apparent to those skilled in the art having the benefit of this disclosure that many more modifications than mentioned above are possible without departing from the inventive concepts disclosed herein.

1. A device for preparing of a mixture of supplements, comprising:
a plurality of ingredient interfaces each couplable to a replaceable container;
a hydraulic system for pumping liquids with metering pumps; and
an electronic controller configured to:
receive an instruction to prepare a mixture based on a general recipe;
detect one or more replaceable containers currently coupled to at least one of the plurality of ingredient interfaces;
determine an ingredient identifier list associated with the one or more replaceable containers and associated properties of ingredients in the ingredient identifier list; and
in response to the instruction, actuate selected metering pumps of the hydraulic system to deliver ingredient volumes from the one or more replaceable containers in accordance with the general recipe and initiate a rinsing flow through the hydraulic system toward an outlet.

2. The device of claim 1, wherein the associated properties include one or more of: physical properties comprising at least viscosity, chemical properties, and a current remaining volumes of the ingredients in the one or more replaceable containers.

3. The device of claim 1, wherein the hydraulic system comprises:
tubing forming a common conduit and branch lines from the metering pumps, the common conduit extending from a water valve to an outlet nozzle disposed at a front portion of the device above a cup platform;
one-way check valves downstream of each respective metering pump to prevent backflow toward the respective metering pump, and additional one-way valves disposed in the common conduit upstream of each junction where a branch line connects to prevent reverse flow into other lines; and
a side-mounted reservoir with a lid, installable and removable by sliding sideways or by pulling up relative to the device.

4. The device of claim 3, wherein the hydraulic system executes:
a cleaning cycle using a special cleaning capsule to flush and sanitize the hydraulic system including the common conduit and the outlet nozzle.

5. The device of claim 1, wherein the selected metering pumps are actuated based on a dosing scheme that indicates a time of actuation of at least one metering pump based on physical properties of a corresponding ingredient.

6. The device of claim 1, wherein each ingredient interface of the plurality of ingredient interfaces comprises:
a pair of piercing needles fixed to a plastic grid that serves as a container holder, the needles projecting into a respective cell of the plastic grid;
wherein the needles are fluidically connected such that a first needle is coupled to a first metering pump to extract liquid from a container and a second needle is coupled to an air path to admit air into the container;
wherein the needles are positioned to engage two internal valves of a cooperating container, one valve providing a liquid outlet and another value providing an air inlet;
cell-level orientation features including lateral grooves in a lower third of each cell that key container insertion in only one orientation relative to the needles; and
a push-to-open cover for each cell, biased by an internal pusher to keep the cell closed in an absence of a container to protect the needles from exposure and accidental contact, the push-to-open cover opens upon being pressed for container insertion.

7. The device of claim 6, wherein the device is compatible with a factory-sealed, single-use container having a parallelepiped body with a lid, an affixed NFC tag, and two internal one-way valves on an inner surface respectively engaged by the needles.

8. The device of claim 1, further comprising:
a structural frame;
a rear, upper internal bracket supporting a power supply;
a rear power inlet;
a rear on/off switch; and
a power cord connecting the rear power inlet to the power supply.

9. The device of claim 8, further comprising:
a printed circuit board mounted within a central space of the structural frame and connected to the power supply at an upper portion;
a body of the device comprises of a container side and a part for installing a water reservoir and covering a central part when the water reservoir is removed;
a front display mounted to the structural frame and surrounded by a snap-fit bezel; and
a cup platform disposed below an outlet nozzle, the cup platform seated in grooves and retained by a weight of the cup platform.

10. The device of claim 1, wherein the electronic controller comprises a plurality of controllers comprising:
a real-time operating system (RTOS) controller on a printed circuit board configured to operation of the metering pumps and a rinse function;
a front display controller in communication with the RTOS controller and configured to issue preparation commands; and
wherein the metering pumps are electrically connected to the printed circuit board by connectors.

11. The device of claim 1, further comprising:
a wireless network interface configured to connect to a Wi-Fi network and a display function configured to present a QR code for pairing the device with a user profile via a mobile application; and
an NFC reader located at a marked area adjacent a container holder and configured to read NFC tags on containers, the device being configured to transmit information about installed containers to a server, to receive and present a server-provided slot assignment for each container, and to receive preparation formulas specifying slot identifiers, pump activation durations, and pump drive voltages.

12. A method for preparing of a mixture of supplements using a dispensing device, comprising:
receiving, by an electronic controller of the dispensing device, an instruction to prepare a mixture based on a general recipe;
detecting, by the electronic controller, one or more replaceable containers currently coupled to at least one of a plurality of ingredient interfaces;
determining, by the electronic controller, an ingredient identifier list associated with the one or more replaceable containers and associated properties of ingredients in the ingredient identifier list; and
in response to the instruction, actuating selected metering pumps of a hydraulic system of the dispensing device to deliver ingredient volumes from the one or more replaceable containers in accordance with the general recipe and initiate a rinsing flow through the hydraulic system toward an outlet of the dispensing device.

13. The method of claim 12, wherein the associated properties include one or more of: physical properties comprising at least viscosity, chemical properties, and a current remaining volumes of the ingredients in the one or more replaceable containers.

14. The method of claim 12, wherein the hydraulic system comprises:
tubing forming a common conduit and branch lines from the metering pumps, the common conduit extending from a water valve to an outlet nozzle disposed at a front portion of the method above a cup platform;
one-way check valves downstream of each respective metering pump to prevent backflow toward the respective metering pump, and additional one-way valves disposed in the common conduit upstream of each junction where a branch line connects to prevent reverse flow into other lines; and
a side-mounted reservoir with a lid, installable and removable by sliding sideways or by pulling up relative to the dispensing device.

15. The method of claim 14, wherein the hydraulic system executes:
a cleaning cycle using a special cleaning capsule to flush and sanitize the hydraulic system including the common conduit and the outlet nozzle.

16. The method of claim 12, wherein the selected metering pumps are actuated based on a dosing scheme that indicates a time of actuation of at least one metering pump based on physical properties of a corresponding ingredient.

17. The method of claim 12, wherein each ingredient interface of the plurality of ingredient interfaces comprises:
a pair of piercing needles fixed to a plastic grid that serves as a container holder, the needles projecting into a respective cell of the plastic grid;
wherein the needles are fluidically connected such that a first needle is coupled to a first metering pump to extract liquid from a container and a second needle is coupled to an air path to admit air into the container;
wherein the needles are positioned to engage two internal valves of a cooperating container, one valve providing a liquid outlet and another value providing an air inlet;
cell-level orientation features including lateral grooves in a lower third of each cell that key container insertion in only one orientation relative to the needles; and
a push-to-open cover for each cell, biased by an internal pusher to keep the cell closed in an absence of a container to protect the needles from exposure and accidental contact, the push-to-open cover opens upon being pressed for container insertion.

18. The method of claim 17, wherein the dispensing device is compatible with a factory-sealed, single-use container having a parallelepiped body with a lid, an affixed NFC tag, and two internal one-way valves on an inner surface respectively engaged by the needles.

19. The method of claim 12, wherein the dispensing device further comprises:
a structural frame;
a rear, upper internal bracket supporting a power supply;
a rear power inlet;
a rear on/off switch; and
a power cord connecting the rear power inlet to the power supply.

20. The method of claim 19, wherein the dispensing device further comprises:
a printed circuit board mounted within a central space of the structural frame and connected to the power supply at an upper portion;
a body of the dispensing device comprises of a container side and a part for installing a water reservoir and covering a central part when the water reservoir is removed;
a front display mounted to the structural frame and surrounded by a snap-fit bezel; and
a cup platform disposed below an outlet nozzle, the cup platform seated in grooves and retained by a weight of the cup platform.
